(19) United States
(12) Patent Application Publication
(10) Pub. No.: US 2025/0125005 A1
(43) Pub. Date: Apr. 17, 2025

(54) VACCINE DESIGN PIPELINE

(71) Applicant: INTOMICS A/S, LYNGBY (DK)

(72) Inventors: Claus LUNDEGAARD, LYNGBY (DK); Juliet WAIRIMU FREDERIKSEN, LYNGBY (DK); Federico DE MASI, LYNGBY (DK)

(73) Assignee: INTOMICS A/S, LYNGBY (DK)

(21) Appl. No.: 18/684,610

(22) PCT Filed: Aug. 17, 2022
(86) PCT No.: PCT/EP2022/072895

§ 371 Date: Feb. 16, 2024

(30) Foreign Application Priority Data
Aug. 17, 2021 (EP) 21191753.9

(51) Int. Cl. G16B 15/30; G16B 15/20; G16B 30/10; G16B 50/30

(52) U.S. Cl. CPC G16B 15/30; G16B 15/20; G16B 30/10; G16B 50/30

(57) ABSTRACT

Herein are provided computer implemented methods for designing sets of peptides, such as for use in a vaccine. Also provided are computer-readable media, computer program products and sets of propagated signals for designing sets of peptides, such as for use in a vaccine. Further provided are methods of treatment, uses and kits comprising peptides designed according to the computer implemented methods.

### CROSS-REFERENCE TO RELATED APPLICATIONS

This application is a U.S. National Stage Application under 35 U.S.C. § 371 of International Patent Application No. PCT/EP2022/072895, filed Aug. 17, 2022, the entire disclosure of which is incorporated by reference herein for all purposes.

### TECHNICAL FIELD

The present disclosure relates to computer implemented methods for designing sets of peptides, such as for use in a vaccine.

### BACKGROUND

Vaccines are probably the most successful medical invention together with antibiotics when it comes to number of saved lives worldwide. A vaccine makes a person resistant (immune) towards a later pathogenic infection by mimicking all or parts of a pathogen.

Vaccines work by taking advantage of antigen recognition and the antibody response. A vaccine contains the antigens of a pathogen that causes disease. When a person is vaccinated, the immune system responds by stimulating antibody-producing cells that are capable of making antigen-specific antibodies. Some of the pre-cursor cells are long lived (so called memory B-cells), that will develop into antibody producing plasma B-cells upon activation by a new encounter of the pathogen. Antibodies can in many cases be detected years after vaccination/infection, either because of very stable antibodies, resident long lived plasma B-cells, or a continuous slow conversion of memory B-cells to plasma B-cells. Similarly, the two types of T-cells, Th and Tc-cells, have long lasting memory precursor cells with the potential to become mature T-cells once its TCR is activated with the proper epitope recognition from the relevant pathogen at a later infection.

Both for safety reasons, and in order to make production-wise more simple vaccines, it has for a long time been a desire to create vaccines consisting only of the parts of the pathogen that are relevant in an immunological context in order to create protective vaccines by sufficient stimulation of the immune response.

Peptides that are able to generate an immune response, such as from a pathogen or comprised in a vaccine, are presented to immune cells on MHC class I or MHC class II molecules. These MHC molecules are in humans termed HLAs (Human Leukocyte Antigens) and are encoded in a large number of allelic variants in the population. Several thousands of alleles have been documented for each of the different loci encoding the HLAs.

The large number of allelic variations is an important problem when it comes to engineering more simple vaccines, as even though HLAs can be divided into so-called supertypes that have overall the same peptide binding pattern, it is by no means ensured that a given peptide will bind to all members of a given supertype. Additionally, the frequencies of the different HLA alleles can be very different between ethnic populations and even between populations of the same ethnical origin that have been separated for a number of generations.

Additionally, it is desirable to create vaccines that protect broadly against all variations of a given pathogen, such that the stimulated immune response is directed towards as many mutants of the pathogenic organism as possible.

There thus exists a large unmet need for designing vaccines that not only are able to elicit a protective immune response directed towards as many variants of a target pathogen as possible, but which are also effective in as large a fraction of a target population as possible, e.g. such as by the vaccine encoding or comprising peptides that can be bound by HLA alleles in as large a part of the target population as possible.

### SUMMARY

Herein is provided a semi-automated in silico method for designing sets of short peptides that are able to elicit efficient immune response directed towards a target pathogen in a large fraction of a target population, the immune response having broad specificity to different strain variants of the pathogen. Additionally, the methods as disclosed herein comprise an extension step for MHC class II binding peptides. This step ensures that the longer peptides better emulate the 3-D structure of the native peptide hosting protein, allowing the peptides to elicit both the T-cell and B-cell response necessary for a sufficient immune response for early clearance and/or protection against the target pathogen.

The computer implemented methods as described herein thus integrate HLA allele population coverage, pathogen protein variance, MHC class I/II binding prediction and intelligent MHC class II binding epitope extension. The resulting output is a unique epitope composition optimised for stimulation of all branches of the adaptive immune system, as well as for optimal coverage of pathogen and human host genetic variance.

The peptides sets are thus designed to be able to efficiently elicit an immune response as broadly as possible across specific populations, by being able to bind to as many human leukocyte antigen (HLA) allelic variants present in the population group as possible. The computer implemented method may optionally comprise designing the peptides to be able to stimulate both parts of the human adaptive immune response, i.e. the humoral and the cellular immune response, by combining both CD4⁺ T cell and antibody epitopes in the same peptide. Several designed peptides may then be incorporated into e.g. a long DNA vaccine construct or an mRNA vaccine construct, or the peptides may be synthesized directly, for use in a vaccine.

In one aspect of the present disclosure is thus provided a computer implemented method for designing a set of peptides, the method comprising the steps of:
1) providing a computer-readable list of protein sequences encoded by a target pathogen genome, wherein
   i. the list further comprises protein sequences encoded by a genome of at least one variant of said target pathogen (variant protein sequences); and
   ii. each protein sequence from a protein that is at least partly extracellular is assigned a computer-readable classifier;
2) aligning said variant protein sequences for each at least partly extracellular protein sequence by multiple alignment and generating a consensus sequence for each extracellular protein;
4) creating a 15-mer peptide set comprising all unique 15-mer peptide sequences for all protein sequences;
6) predicting MHC class II binding for each unique 15-mer peptide, for at least one MHC class II allele, such as for at least one HLA allele selected from the group consisting of HLA-DP, HLA-DQ and HLA-DR;
7) creating a first set of selected peptides, wherein the first set of selected peptides comprises the unique 15-mer peptides that are predicted to bind to at least one MHC class II allele, such as at least one HLA allele, with a minimum binding score;
8) optionally, validating the immunogenicity of one or more peptides from the first set of peptides, such as by an in vivo assay, an in vitro assay and/or by database lookup, thereby generating a first set of validated peptides, 9) combining data describing
   i. the first set of selected peptides;
   ii. the corresponding MHC class II alleles predicted to bind each peptide in the first set of selected peptides; and
   iii. MHC class II allele frequencies in a target population,
   or, if step 8) has been performed,
   i. the first set of validated peptides;
   ii. the corresponding MHC class II alleles predicted to bind each peptide in the first set of validated peptides; and
   iii. MHC class II allele frequencies in a target population,
   to generate a second set of selected peptides, wherein the second set of peptides comprises peptides that, when taken together, are
   i. present in at least 75%, such as at least 80%, such as at least 85%, such as at least 90%, or such as at least 95% of said variants of said target pathogen; and
   ii. predicted to be bound by at least one MHC class II allele present in said target population, in at least 75%, such as at least 80%, such as at least 85%, such as at least 90%, or such as in at least 95% of said target population;
10) creating a third set of peptides from the second set of selected peptides by
   i. extending the 15-mer peptides that originate from proteins classified as at least partly extracellular using the consensus sequence generated for each protein in step 2) in the N- and C-terminal directions until the peptide length is between 25 to 35 amino acids, such as between 28 to 32 amino acids, such as 30 amino acids, thereby creating the third set of peptides comprising MHC class II binding peptides and/or extended MHC class II binding peptides; or
   ii. for each 15-mer peptide, determining the corresponding full-length variant protein sequence of step 1) with the highest sequence identity to the consensus sequence generated in step 2) and extending each 15-mer peptide with the determined corresponding full-length protein sequence that flanks the 15-mer peptide sequence to create one or more mosaic protein sequences, thereby creating the third set of peptides comprising MHC class II binding peptides and/or mosaic protein sequences.

In some aspects, the present disclose provides a method for producing and formulating a vaccine, said method comprising the steps of:
1) performing the method as disclosed herein; and
2) producing and formulating at least one peptide from the third set of peptides and/or a nucleic acid sequence encoding said peptide.

In some aspects of the present disclosure is provided a computer program product comprising instructions which, when the program is executed by a computer, cause the computer to carry out the method as described herein.

In some aspects is provided a computer-readable medium comprising instructions which, when executed by a computer, cause the computer to carry out the method as described herein.

In some aspects of the present disclosure is provided a set of propagated signals comprising computer readable instructions which, when executed by a computer, cause the computer to carry out the method as described herein.

In some aspects is provided a data processing system comprising a processor configured to perform the method as described herein.

In some aspects of the present disclosure is thus provided a composition comprising one or more peptides or one or more nucleic acids encoding said one or more peptides, wherein the one or more peptides are designed using to the method as described herein.

In some aspects is provided a pharmaceutical composition comprising one or more peptides or one or more nucleic acids encoding said one or more peptides, wherein the one or more peptides are designed using to the method as described herein, and a pharmaceutically acceptable diluent, carrier and/or excipient.

In some aspects is provided the use of a peptide or a nucleic acid encoding said peptide, wherein the peptide is designed according to the methods as disclosed elsewhere herein, in the prophylaxis and/or treatment of a disease.

In some aspects is provided a peptide, or a nucleic acid encoding said peptide, designed according to the methods as disclosed herein for use in a method for treating and/or preventing a disease in a subject.

In some aspects is provided a method for treating and/or preventing a disease in a subject in need thereof, the method comprising administering to the subject a pharmaceutical composition as disclosed elsewhere herein.

In some aspects of the present disclosure is provided a kit of parts comprising:
1) a composition or a pharmaceutical composition, such as a vaccine, as defined elsewhere herein; and
2) optionally, a medical instrument or other means for administering the composition; and
3) instructions for use.

### DESCRIPTION OF DRAWINGS

FIG. 1 shows an overview of the Influenza A virus structure and its various surface Proteins. As can be seen in the figure, HA, NA, and M2 are membrane proteins with at least a domain in the extracellular space.

FIG. 2 shows an example of generating a consensus sequence from a membrane protein with at least a domain in the extracellular space. All variants of each protein from proteins assigned to have at least a domain in the extracellular space are aligned by a multiple alignment method, e.g., CLUSTALW or MAFFT. For each protein, a consensus sequence is generated, e.g., using the most abundant amino acid at a given position. The consensus sequence shown in the figure corresponds to SEQ ID NO: 1.

FIG. 3 shows an overview of the steps in the pipeline according to the present disclosure with example algorithms listed for each relevant step.

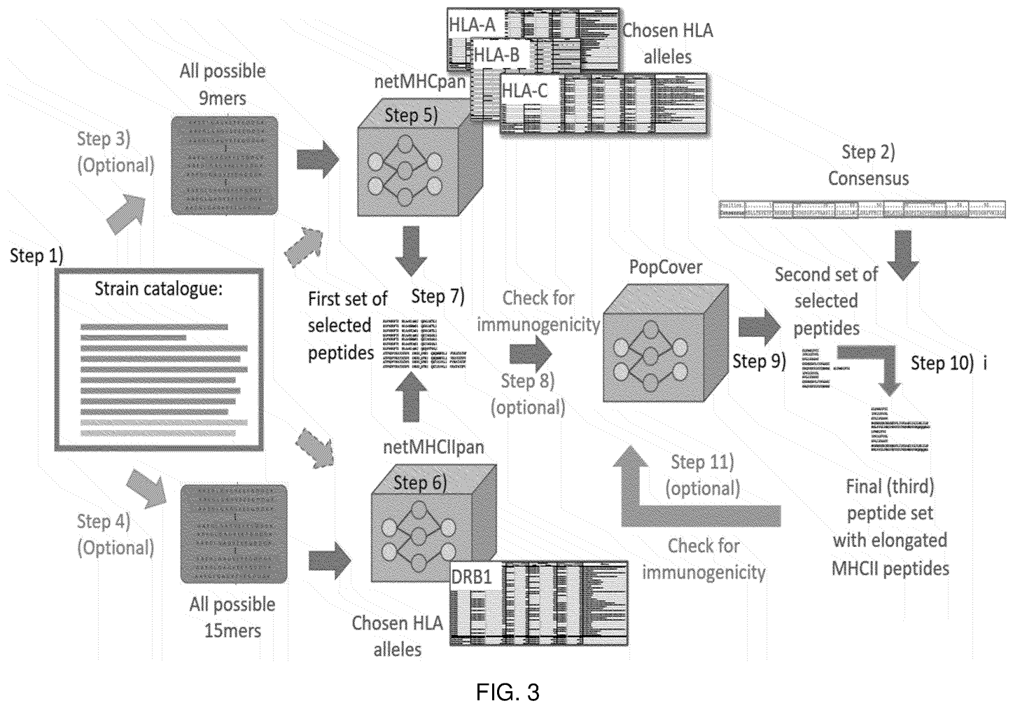

FIG. 4 shows the coverage of epitope sets with increasing number of epitopes.

Selected peptides are added to the set in the order selected by method described in Examples 3 and 4. Vaccine epitopes are selected randomly between all available epitopes with 100 different random choices in each step. The coverage is shown as the average of the 100 choices and the standard deviation is depicted.

### DETAILED DESCRIPTION

### Definitions

The term “at least partly extracellular protein” as used herein refers to:
a. a protein excreted into the extracellular environment of the infected host by the targeted pathogen;
b. a protein excreted into the extracellular environment by a host cell infected by the pathogen;
c. a membrane protein of the targeted pathogen with at least 15 amino acids being on the outer side of the pathogen's outer membrane/cell wall; and/or
d. a protein integrated in the capsid of the targeted pathogen with at least 15 amino acids being accessible from the outside of the pathogen,
   preferably wherein said protein is important for establishing and/or maintaining an infection caused by the targeted pathogen.

The term “at least partly extracellular protein” may further refer to a pathogen protein that is at least partly located in an extracellular compartment, such as on the surface of a cell, or a viral particle at least partly located in an extracellular compartment. The protein may be fully extracellular (e.g. such as a viral capsid protein or a bacterial surface protein), but may also be a transmembrane protein (e.g. such as the M2 protein of influenza A). Transmembrane proteins comprise both a part of the protein that is located extracellularly or on the surface of a particle and a part of the protein that is located intracellularly or inside the particle, and thus also falls under the definition of the term.

### Computer-Implemented Methods for Designing a Set of Peptides

Herein is a provided a semi-automated in silico pipeline for designing sets of short peptides predicted to be able to elicit an immune response directed towards a target pathogen in a large part of a target population, the immune response having broad specificity to different strain variants of the pathogen.

In one aspect of the present disclosure is thus provided a computer implemented method for designing a set of peptides, the method comprising the steps of:
1) providing a computer-readable list of protein sequences encoded by a target pathogen genome, wherein
   i. the list further comprises protein sequences encoded by a genome of at least one variant of said target pathogen (variant protein sequences); and
   ii. each protein sequence from a protein that is at least partly extracellular is assigned a computer-readable classifier;
2) aligning said variant protein sequences for each at least partly extracellular protein sequence by multiple alignment and generating a consensus sequence for each extracellular protein;
4) creating a 15-mer peptide set comprising all unique 15-mer peptide sequences for all protein sequences;
6) predicting MHC class II binding for each unique 15-mer peptide, for at least one MHC class II allele, such as for at least one HLA allele selected from the group consisting of HLA-DP, HLA-DQ and HLA-DR;
7) creating a first set of selected peptides, wherein the first set of selected peptides comprises the unique 15-mer peptides that are predicted to bind to at least one MHC class II allele, such as at least one HLA allele, with a minimum binding score;
8) optionally, validating the immunogenicity of one or more peptides from the first set of peptides, such as by an in vivo assay, an in vitro assay and/or by database lookup, thereby generating a first set of validated peptides,
9) combining data describing
   i. the first set of selected peptides;
   ii. the corresponding MHC class II alleles predicted to bind each peptide in the first set of selected peptides; and
   iii. MHC class II allele frequencies in a target population,
   or, if step 8) has been performed,
   i. the first set of validated peptides;
   ii. the corresponding MHC class II alleles predicted to bind each peptide in the first set of validated peptides; and
   iii. MHC class II allele frequencies in a target population,
   to generate a second set of selected peptides, wherein the second set of peptides comprises peptides that, when taken together, are
   i. present in at least 75%, such as at least 80%, such as at least 85%, such as at least 90%, or such as at least 95% of said variants of said target pathogen; and
   ii. predicted to be bound by at least one MHC class II allele present in said target population, in at least 75%, such as at least 80%, such as at least 85%, such as at least 90%, or such as in at least 95% of said target population;
10) creating a third set of peptides from the second set of selected peptides by
   i. extending the 15-mer peptides that originate from proteins classified as at least partly extracellular using the consensus sequence generated for each protein in step 2) in the N- and C-terminal directions until the peptide length is between 25 to 35 amino acids, such as between 28 to 32 amino acids, such as 30 amino acids, thereby creating the third set of peptides comprising MHC class II binding peptides and/or extended MHC class II binding peptides; or
   ii. for each 15-mer peptide, determining the corresponding full-length variant protein sequence of step 1) with the highest sequence identity to the consensus sequence generated in step 2) and extending each 15-mer peptide with the determined corresponding full-length protein sequence that flanks the 15-mer peptide sequence to create one or more mosaic protein sequences, thereby creating the third set of peptides comprising MHC class II binding peptides and/or mosaic protein sequences.

In some embodiments is provided a computer implemented method for designing a set of peptides, the method comprising the steps of:
1) providing a computer-readable list of protein sequences encoded by a target pathogen genome, wherein
   i. the list further comprises protein sequences encoded by a genome of at least one variant of said target pathogen (variant protein sequences); and
   ii. each protein sequence from a protein that is at least partly extracellular is assigned a computer-readable classifier;
2) aligning said variant protein sequences for each at least partly extracellular protein sequence by multiple alignment and generating a consensus sequence for each extracellular protein;
3) optionally, creating an 8-11-mer peptide set comprising all unique 8-, 9-, 10- and/or 11-mer peptide sequences for all protein sequences;
4) creating a 15-mer peptide set comprising all unique 15-mer peptide sequences for all protein sequences;
5) optionally, predicting MHC class I binding for each unique 8-11-mer peptide, for at least one MHC class I allele, such as for at least one human leukocyte antigen (HLA) allele selected from the group consisting of HLA-A, HLA-B and HLA-C;
6) predicting MHC class II binding for each unique 15-mer peptide, for at least one MHC class II allele, such as for at least one HLA allele selected from the group consisting of HLA-DP, HLA-DQ and HLA-DR;
7) creating a first set of selected peptides, wherein the first set of selected peptides comprises the unique 8-11-mer peptides and/or the unique 15-mer peptides that are predicted to bind to at least one MHC class I and/or MHC class II allele, respectively, with a minimum binding score;
8) optionally, validating the immunogenicity of one or more peptides from the first set of peptides, such as by an in vivo assay, an in vitro assay and/or by database lookup, thereby generating a first set of validated peptides,
9) combining data describing
   i. the first set of selected peptides;
   ii. the corresponding MHC class I and/or MHC class II alleles predicted to bind each peptide in the first set of selected peptides; and
   iii. MHC class I and/or MHC class II allele frequencies in a target population,
   or, if step 8) has been performed,
   i. the first set of validated peptides;
   ii. the corresponding MHC class I and/or MHC class II alleles predicted to bind each peptide in the first set of validated peptides; and
   iii. MHC class I and/or MHC class II allele frequencies in a target population,
   to generate a second set of selected peptides, wherein the second set of peptides comprises peptides that, when taken together, are
   i. present in at least 75%, such as at least 80%, such as at least 85%, such as at least 90%, or such as at least 95% of said variants of said target pathogen; and
   ii. predicted to be bound by at least one MHC class I and/or MHC class II allele present in said target population, in at least 75%, such as at least 80%, such as at least 85%, such as at least 90%, or such as in at least 95% of said target population;
10) creating a third set of peptides from the second set of selected peptides by
   i. extending the 15-mer peptides that originate from proteins classified as at least partly extracellular using the consensus sequence generated for each protein in step 2) in the N- and C-terminal directions until the peptide length is between 25 to 35 amino acids, such as between 28 to 32 amino acids, such as 30 amino acids, thereby creating the third set of peptides comprising MHC class II binding peptides and/or extended MHC class II binding peptides; or
   ii. for each 15-mer peptide, determining the corresponding full-length variant protein sequence of step 1) with the highest sequence identity to the consensus sequence generated in step 2) and extending each 15-mer peptide with the determined corresponding full-length protein sequence that flanks the 15-mer peptide sequence to create one or more mosaic protein sequences, thereby creating the third set of peptides comprising MHC class II binding peptides and/or mosaic protein sequences.

In some embodiments, the method further comprises a step 11) of validating the immunogenicity of one or more peptides from the third set of peptides, such as by an in vivo assay, an in vitro assay and/or by database lookup, thereby generating a second set of validated peptides, and optionally repeating steps 8) to 9) using said second set of validated peptides.

Said in vitro assay may be an assay such as those described in Example 2 herein.

Said in vivo assay may be an assay measuring a T-cell and/or antibody response of one or more peptides from the third set of peptides, such as the assay described in Ewer et al., 2021.

Relevant databases for validating the immunogenicity of the peptides in the third set of peptides include The Immune Epitope Database (IEDB) (https://eee.iedb.org) as also described in Vita et al., 2019.

Step 1) of the method as described herein comprises providing a computer-readable list of protein sequences encoded by a target pathogen genome, said list further comprising protein sequences encoded by a genome of at least one variant of said target pathogen (also referred to herein as variant protein sequences) and each protein sequence in the list that originates from a protein that is at least partly extracellular being assigned a computer-readable classifier. Said list may be provided in any way or format that can be read by a computer. In some embodiments, said computer-readable list is provided as a text file. In some embodiments, said computer-readable list is provided through a user interface. In some embodiments, said computer-readable list is read from a database. In some embodiments, said computer-readable list is read from a website.

In some embodiments, proteins that, when present in the extracellular space, are known to not be important for establishment or maintenance of an infection are not classified as at least partly extracellular, or the classifier marking the proteins as at least partly extracellular may be manually removed. This may be the case even for proteins that are expressed on the surface of the infected cell or are exported, and which would otherwise be classified as at least partly extracellular. This step may simplify the vaccine design in cases where full-length or elongated peptides from certain proteins will not give any benefits, as antibodies against the given proteins will not clear the pathogen or limit infection.

In some embodiments, the provided computer-readable list of protein sequences of step 1) comprises or consists of unique 8-11-mer and/or 15-mer peptide sequences. In some embodiments said unique 8-11-mer and/or 15-mer peptide sequences are annotated peptide epitopes. If such a list of unique 8-11-mer and/or 15-mer peptide sequences is provided, the steps of creating 8-11-mer and/or 15-mer peptide sets (steps 3 and/or 4, respectively) may be skipped.

Step 2) of the method as described herein comprises a step of performing a multiple alignment of the variant peptide sequences provided in step 1) to generate a consensus sequence, wherein the consensus sequence for each extracellular protein is generated using the most abundant amino acid at a given position.

Said multiple alignment may be performed using any of the multiple alignment methods known to the skilled person. In some embodiments, the multiple alignment of step 2) of the method as described herein is performed using CLUSTALW. In some embodiments, the multiple alignment of step 2) of the method as described herein is performed using MAFFT.

Step 3) of the method as described herein is optional and comprises creating a peptide set comprising all unique 8-, 9-, 10- and/or 11-mer peptide sequences for all protein sequences provided.

The list of all 8-, 9-, 10- and/or 11-mer peptide sequences comprises all unique 8-mer peptide sequences, all unique 9-mer peptide sequences, all unique 10-mer peptide sequences and/or all unique 11-mer peptide sequences. In some embodiments, the 8-11-mer peptide set comprises all unique 8-mer peptide sequences. In some embodiments, the 8-11-mer peptide set comprises all unique 9-mer peptide sequences. In some embodiments, the 8-11-mer peptide set comprises all unique 10-mer peptide sequences. In some embodiments, the 8-11-mer peptide set comprises all unique 11-mer peptide sequences. In some embodiments, the 8-11-mer peptide set comprises all unique 8-mer peptide sequences and all unique 9-mer peptide sequences. In some embodiments, the 8-11-mer peptide set comprises all unique 9-mer peptide sequences and all unique 10-mer peptide sequences. In some embodiments, the 8-11-mer peptide set comprises all unique 10-mer peptide sequences and all unique 11-mer peptide sequences. In some embodiments, the 8-11-mer peptide set comprises all unique 8-mer peptide sequences, all unique 9-mer peptide sequences and all unique 10-mer peptide sequences. In some embodiments, the 8-11-mer peptide set comprises all unique 9-mer peptide sequences, all unique 10-mer peptide sequences and all unique 11-mer peptide sequences. In some embodiments, the 8-11-mer peptide set comprises all unique 8-mer peptide sequences, all unique 9-mer peptide sequences, all unique 10-mer peptide sequences and all unique 11-mer peptide sequences.

It may be useful to link certain information together with each 8-11-mer peptide sequence, such as the protein identifier and/or strain information from which the peptide sequence originated. Thus, in some embodiments, the 8-11-mer peptides of step 3) are digitally stored with origin strain information for use in step 9).

Step 4) of the method as described herein comprises predicting of the method as described herein comprises creating a peptide set comprising all unique 15-mer peptide sequences for all protein sequences provided.

It may be useful to link certain information together with each 15-mer peptide sequence, such as the protein identifier and/or strain information from which the peptide sequence originated. Thus, in some embodiments, the 15-mer peptides of step 4) are digitally stored with origin strain information for use in step 9).

Step 5) of the method as described herein is optional and comprises predicting MHC class I binding for each unique 8-11-mer peptide either provided as part of the computer-readable list in step 1 or created in step 3, for at least one allele encoding an MHC class I allele. Said MHC class I allele may be a human leukocyte antigen (HLA) corresponding to MHC class I, such as at least one HLA allele selected from the group consisting of HLA-A, HLA-B and HLA-C. Said MHC class I binding may be predicted using any useful method known in the art, such as those described in Phloyphisut et al., 2019.

In some embodiments, predicting MHC class I binding for each unique 8-11-mer peptide in step 5) is performed using an algorithm selected from the list consisting of NetMHCpan, MHCSeqNet, NetMHC, NetMHCcons, PickPocket and MHCflurry.

In some embodiments, predicting MHC class I binding for each unique 8-11-mer peptide in step 5) is performed using MHCSeqNet. In some embodiments, predicting MHC class I binding for each unique 8-11-mer peptide in step 5) is performed using NetMHC, such as NetMHC version 3.4, or such as NetMHC version 4.0. In some embodiments, predicting MHC class I binding for each unique 8-11-mer peptide in step 5) is performed using NetMHCcons, such as NetMHCcons version 1.1. In some embodiments, predicting MHC class I binding for each unique 8-11-mer peptide in step 5) is performed using PickPocket, such as PickPocket version 1.1. In some embodiments, predicting MHC class I binding for each unique 8-11-mer peptide in step 5) is performed using MHCflurry, such as MHCflurry version 1.2.

In preferred embodiments, predicting MHC class I binding for each unique 8-11-mer peptide in step 5) is performed using NetMHCpan, such as NetMHCpan version 2.8, such as NetMHCpan version 3.0, such as NetMHCpan version 4.0 or such as NetMHCpan version 4.1.

Step 6) of the method as described herein comprises predicting MHC class II binding for each unique 15-mer peptide either provided as part of the computer-readable list in step 1 or created in step 4, for at least one allele encoding an MHC class II allele. Said MHC class II allele may be a human leukocyte antigen (HLA) corresponding to MHC class II, such as a HLA allele selected from the group consisting of HLA-DP, HLA-DQ and HLA-DR. Said MHC class II binding may be predicted using any useful method known in the art, such as those described in Chen et al., 2019 and Zhang et al., 2019.

In some embodiments, predicting MHC class II binding for each unique 15-mer peptide in step 6) is performed using an algorithm selected from the list consisting of NetMHCIIpan, MARIA and MoDec.

In some embodiments, predicting MHC class II binding for each unique 15-mer peptide in step 6) is performed using MARIA. In some embodiments, predicting MHC class II binding for each unique 15-mer peptide in step 6) is performed using MoDec.

In preferred embodiments, predicting MHC class II binding for each unique 15-mer peptide in step 6) is performed using NetMHCIIpan, such as NetMHCIIpan version 4.0.

It may be useful to link certain information together with each 8-11-mer and 15-mer peptide sequence and their respective predicted MHC class I or MHC class II molecule binding strength, such as the protein identifier and/or strain information from which the peptide sequence originated. Thus, in some embodiments the predicted MHC class I and/or MHC class II binding of each peptide of steps 5) and 6) is digitally stored with origin strain information and digitally formatted for use in step 9).

Step 7) of the method as described herein comprises creating a first set of selected peptides, said list comprising unique 8-11-mer and/or 15-mer peptides that are predicted in steps 5 and/or 6 to bind to at least one MHC class I or II allele, such as at least one HLA allele, with a minimum binding score. Said minimum binding score is set to ensure a high probability that each selected peptide can bind to at least one of the selected MHC class I or II alleles, such as at least one of the selected HLA alleles, in vivo. As will be readily apparent to the skilled person, said binding score may change according to the method used to assess binding strength in step 5 and/or 6, i.e. said binding score is method dependent.

In some embodiments, the minimum binding score of step 7) is defined as a minimum affinity threshold. In some embodiments, said minimum affinity threshold is 1 μM. In some embodiments, said minimum affinity threshold is 900 nM. In some embodiments, said minimum affinity threshold is 800 nM. In some embodiments, said minimum affinity threshold is 700 nM. In some embodiments, said minimum affinity threshold is 600 nM. In some embodiments, said minimum affinity threshold is 500 nM. In some embodiments, said minimum affinity threshold is 400 nM. In some embodiments, said minimum affinity threshold is 300 nM. In some embodiments, said minimum affinity threshold is 200 nM. In some embodiments, said minimum affinity threshold is 100 nM. In some embodiments, said minimum affinity threshold is 50 nM. In some embodiments, said minimum affinity threshold is 25 nM. In some embodiments, said minimum affinity threshold is 20 nM. In some embodiments, said minimum affinity threshold is 10 nM. In some embodiments, said minimum affinity threshold is 5 nM. In some embodiments, said minimum affinity threshold is 2 nM. In some embodiments, said minimum affinity threshold is 1 nM.

In some embodiments, the minimum binding score of step 7) is defined as a minimum rank threshold. Thus, only the top ranking peptides, i.e. a certain percentage of peptides with the highest predicted binding score, may be selected in step 7.

In some embodiments, the minimum rank threshold is the top 5%. In some embodiments, the minimum rank threshold is the top 4%. In some embodiments, the minimum rank threshold is the top 3%. In some embodiments, the minimum rank threshold is the top 2%. In some embodiments, the minimum rank threshold is the top 1%. In some embodiments, the minimum rank threshold is the top 0.5%.

In some embodiments, the binding score is predicted using NetMHCpan-4.1 and the minimum rank threshold is the top 2%. In some embodiments, the minimum binding score is predicted using NetMHCpan-4.1 and the minimum rank threshold is the top 1.5%. In some embodiments, the minimum binding score is predicted using NetMHCpan-4.1 and the minimum rank threshold is the top 1%. In some embodiments, the minimum binding score is predicted using NetMHCpan-4.1 and the minimum rank threshold is the top 0.5%.

In some embodiments, the binding score is predicted using NetMHCIIpan-4.0 and the minimum rank threshold is the top 5%. In some embodiments, the binding score is predicted using NetMHCIIpan-4.0 and the minimum rank threshold is the top 4%. In some embodiments, the binding score is predicted using NetMHCIIpan-4.0 and the minimum rank threshold is the top 3%. In some embodiments, the binding score is predicted using NetMHCIIpan-4.0 and the minimum rank threshold is the top 2%. In some embodiments, the binding score is predicted using NetMHCIIpan-4.0 and the minimum rank threshold is the top 1%.

In some embodiments, the minimum binding score of step 7) is defined as a minimum output score threshold. The minimum output score threshold is a minimum value from the output of the binding prediction step(s) that must be exceeded for each selected peptide. Said minimum output score is method dependent.

Step 8) of the method as described herein is optional and comprises validating the immunogenicity of one or more peptides from the first set of peptides, such as by an in vivo assay, an in vitro assay and/or by database lookup, thereby generating a first set of validated peptides. Relevant in vivo and in vitro assays, and database are described herein above.

As not all MHC class I or MHC class II binding peptides are immunogenic in vivo, this step may ensure that only peptides with sufficient immunogenicity are used for the further steps of the method.

Step 9) of the method as described herein comprises combining data describing
i. the first set of selected peptides;
ii. the corresponding MHC class I and/or MHC class II alleles, such as HLA alleles, predicted to bind each peptide in the first set of selected peptides; and
iii. MHC class I and/or MHC class II allele frequencies, such as HLA allele frequencies, in a target population,
   to generate a second set of selected peptides, wherein the second set of peptides comprises peptides that, when taken together, are
i. present in at least 75% of said variants of said target pathogen, such as in at least 80% of said variants of said target pathogen, such as in at least 85% of said variants of said target pathogen, such as in at least 90% of said variants of said target pathogen, or such as in at least 95% of said variants of said target pathogen; and
ii. predicted to be bound by at least one MHC class I or MHC class II, such as a HLA allele, present in said target population, in at least 75% of said target population, such as in at least 80% of said target population, such as in at least 85% of said target population, such as in at least 90% of said target population, or such as in at least 95% of said target population.

If the optional step 8) has been performed then step 9) of the method as described herein instead comprises combining data describing
i. the first set of validated peptides;
ii. the corresponding MHC class I and/or MHC class II alleles, such as HLA alleles, predicted to bind each peptide in the first set of validated peptides; and
iii. MHC class I and/or MHC class II allele frequencies, such as HLA allele frequencies, in a target population,
   to generate a second set of selected peptides, wherein the second set of peptides comprises peptides that, when taken together, are
i. present in at least 75%, such as in at least 80% of said variants of said target pathogen, such as in at least 85% of said variants of said target pathogen, such as in at least 90% of said variants of said target pathogen, or such as in at least 95% of said variants of said target pathogen; and
ii. predicted to be bound by at least one MHC class I or MHC class II alleles, such as HLA alleles, present in said target population, in at least 75% of said target population, such as in at least 80% of said target population, such as in at least 85% of said target population, such as in at least 90% of said target population, or such as in at least 95% of said target population;

This step thus combines the MHC class I and/or MHC class II allele frequencies, such as HLA frequencies, of a selected target population with the MHC class I and/or MHC class II alleles, such as HLA alleles, predicted to be bound by each peptide of either the first selected set of peptides or the first validated set of peptides, in order to generate a second set of selected peptides that covers as much of the pathogen's variation as possible and as much of the selected population MHC class I and/or MHC class II allele diversity, such as HLA allele diversity, as possible. This ensures that the second set of selected peptides has both optimal host coverage and optimal pathogen variant coverage.

In some embodiments, the target population is a mammalian target population, such as a primate target population, a rodent target population, or a mustelid target population.

In some embodiments, the target population is a human target population.

The HLA allele frequencies in a human target population used to calculate HLA allele coverage may be selected for single or combined populations. For example, the target human population may be North Americans and South Americans, or the target human population may be people of Asian descent. In some embodiments, the HLA allele frequencies in a human target population is determined using the allelefrequencies.net database (described in Middleton et al., 2003). In some embodiments, the HLA allele frequencies in a human target population is determined using The Immune Epitope Database (IEDB) (http://tools.iedb.org/population/help/) (described in Vita et al., 2019).

In some embodiments, the second set of selected peptides comprises peptides that, when taken together, are present in at least 75% of said variants of said target pathogen, such as at least 76% of said variants of said target pathogen, such as at least 77% of said variants of said target pathogen, such as at least 78% of said variants of said target pathogen, such as at least 79% of said variants of said target pathogen, such as at least 80% of said variants of said target pathogen, such as at least 81% of said variants of said target pathogen, such as at least 82% of said variants of said target pathogen, such as at least 83% of said variants of said target pathogen, such as at least 84% of said variants of said target pathogen, such as at least 85% of said variants of said target pathogen, such as at least 86% of said variants of said target pathogen, such as at least 87% of said variants of said target pathogen, such as at least 88% of said variants of said target pathogen, such as at least 89% of said variants of said target pathogen, such as at least 90% of said variants of said target pathogen, such as at least 91% of said variants of said target pathogen, such as at least 92% of said variants of said target pathogen, such as at least 93% of said variants of said target pathogen, such as at least 94% of said variants of said target pathogen, such as at least 95% of said variants of said target pathogen, such as at least 96% of said variants of said target pathogen, such as at least 97% of said variants of said target pathogen, such as at least 98% of said variants of said target pathogen, or such as at least 99% of said variants of said target pathogen.

In other words, the second set of selected peptides is optimized to comprise peptides that, when taken together as a set, can be found in or covers at least 75% of all variants of the target pathogen, such as at least 80% of all variants of the target pathogen, such as at least 85% of all variants of the target pathogen, such as at least 90% of all variants of the target pathogen, such as at least 95% of all variants of the target pathogen. This does therefore not mean that the set must comprise peptides wherein each individual peptide is found in at least 75% of target pathogen variants.

In some embodiments, the second set of selected peptides comprises peptides that are predicted to be bound by at least one MHC class I or MHC class II allele present in the target population, such as in at least 75% of the target population, such as in at least 76% of the target population, such as in at least 77% of the target population, such as in at least 78% of the target population, such as in at least 79% of the target population, such as in at least 80% of the target population, such as in at least 81% of the target population, such as in at least 82% of the target population, such as in at least 83% of the target population, such as in at least 84% of the target population, such as in at least 85% of the target population, such as in at least 86% of the target population, such as in at least 87% of the target population, such as in at least 88% of the target population, such as at least 89%, such as in at least 90% of the target population, such as in at least 91% of the target population, such as in at least 92% of the target population, such as in at least 93% of the target population, such as in at least 94% of the target population, such as in at least 95% of the target population, such as in at least 96% of the target population, such as in at least 97% of the target population, such as in at least 98% of the target population, or such as in at least 99% of the target population.

In some embodiments, the second set of selected peptides comprises peptides that are predicted to be bound by at least one HLA allele present in the human target population, such as in at least 75% of the human target population, such as in at least 76% of the human target population, such as in at least 77% of the human target population, such as in at least 78% of the human target population, such as in at least 79% of the human target population, such as in at least 80% of the human target population, such as in at least 81% of the human target population, such as in at least 82% of the human target population, such as in at least 83% of the human target population, such as in at least 84% of the human target population, such as in at least 85% of the human target population, such as in at least 86% of the human target population, such as in at least 87% of the human target population, such as in at least 88% of the human target population, such as at least 89%, such as in at least 90% of the human target population, such as in at least 91% of the human target population, such as in at least 92% of the human target population, such as in at least 93% of the human target population, such as in at least 94% of the human target population, such as in at least 95% of the human target population, such as in at least 96% of the human target population, such as in at least 97% of the human target population, such as in at least 98% of the human target population, or such as in at least 99% of the human target population.

In other words, the second set of selected peptides is optimized to comprise peptides that are predicted to be bound by MHC class I or class II alleles, such as HLA alleles, of the target population. This does not necessarily mean that the set must comprise individual peptides wherein each one peptide is able to bind to as many MHC class I or class II alleles, such as HLA alleles, of the target population as possible. Rather, the second set of selected peptides may comprise individual peptides that, when taken together as a set, are predicted to bind to at least one MHC class I or class II allele, such as at least one HLA allele, in each person in the target population, such as in at least 75% of the target population, such as in at least 80% of the target population, such as in at least 85% of the target population, such as in at least 90% of the target population, or such as in at least 95% of said target population.

In some embodiments, the second set of selected peptides comprises MHC class I allele-binding peptides that are predicted to be bound by at least one MHC class I allele present in the target population, in at least 75% of the target population, such as in at least 76% of the target population, such as in at least 77% of the target population, such as in at least 78% of the target population, such as in at least 79% of the target population, such as in at least 80% of the target population, such as in at least 81% of the target population, such as in at least 82% of the target population, such as in at least 83% of the target population, such as in at least 84% of the target population, such as in at least 85% of the target population, such as in at least 86% of the target population, such as in at least 87% of the target population, such as in at least 88% of the target population, such in as at least 89% of the target population, such as in at least 90% of the target population, such as in at least 91% of the target population, such as in at least 92% of the target population, such as in at least 93% of the target population, such as in at least 94% of the target population, such as in at least 95% of the target population, such as in at least 96% of the target population, such as in at least 97% of the target population, such as in at least 98% of the target population, or such as in at least 99% of the target population.

In some embodiments, the second set of selected peptides comprises MHC class II allele-binding peptides that are predicted to be bound by at least one MHC class II allele present in the target population, such as in at least 75% of the target population, such as in at least 76% of the target population, such as in at least 77% of the target population, such as in at least 78% of the target population, such as in at least 79% of the target population, such as in at least 80% of the target population, such as in at least 81% of the target population, such as in at least 82% of the target population, such as in at least 83% of the target population, such as in at least 84% of the target population, such as in at least 85% of the target population, such as in at least 86% of the target population, such as in at least 87% of the target population, such as in at least 88% of the target population, such as at least 89%, such as in at least 90% of the target population, such as in at least 91% of the target population, such as in at least 92% of the target population, such as in at least 93% of the target population, such as in at least 94% of the target population, such as in at least 95% of the target population, such as in at least 96% of the target population, such as in at least 97% of the target population, such as in at least 98% of the target population, or such as in at least 99% of the target population.

In some embodiments, said MHC class I allele-binding peptides are HLA allele-binding peptides and said target population is a human target population.

In some embodiments, said MHC class II allele-binding peptides are HLA allele-binding peptides and said target population is a human target population.

In some embodiments, said MHC class I allele frequencies in said target population comprise or consist of HLA-A, HLA-B and/or HLA-C allele frequencies in a human target population.

In some embodiments, said MHC class II allele frequencies in said target population comprise or consist of HLA-DP, HLA-DQ and/or HLA-DR allele frequencies in a human target population.

In some embodiments, said target population comprises at least two different species.

In some embodiments, the second set of selected peptides comprises MHC class I and MHC class II allele-binding peptides that are predicted to be bound by, respectively, at least one MHC class I allele or at least one MHC class II allele present in the target population, such as in at least 75% of the target population, such as in at least 76% of the target population, such as in at least 77% of the target population, such as in at least 78% of the target population, such as in at least 79% of the target population, such as in at least 80% of the target population, such as in at least 81% of the target population, such as in at least 82% of the target population, such as in at least 83% of the target population, such as in at least 84% of the target population, such as in at least 85% of the target population, such as in at least 86% of the target population, such as in at least 87% of the target population, such as in at least 88% of the target population, such as at least 89%, such as in at least 90% of the target population, such as in at least 91% of the target population, such as in at least 92% of the target population, such as in at least 93% of the target population, such as in at least 94% of the target population, such as in at least 95% of the target population, such as in at least 96% of the target population, such as in at least 97% of the target population, such as in at least 98% of the target population, or such as in at least 99% of the target population.

In some embodiments, said MHC class I and MHC class II allele-binding peptides are HLA allele-binding peptides and said target population is a human target population.

In some embodiments, the second set of selected peptides of step 9) is generated using the PopCover algorithm (Buggert et al., 2012), such as PopCover-2.0 (https://services.healthtech.dtu.dk/service.php?PopCover-2.0).

In some embodiments, the second set of selected peptides of step 9) is stored with all relevant meta-data in an independent digital table or database. This may include, for each peptide, identifiers for variant of origin, the full sequence of the protein from which the peptide originates, a list of MHC class I and/or MHC class II alleles, such as HLA alleles, bound by the peptide and the frequencies of the MHC class I and/or MHC class II alleles, such as HLA alleles, in a given population.

Step 10) of the method as described herein comprises creating a third set of peptides from the second set of selected peptides by
i. extending the 15-mer peptides that originate from proteins classified as at least partly extracellular using the consensus sequence generated for each protein in step 2) in the N- and C-terminal directions until the peptide length is between 25 to 35 amino acids, such as between 28 to 32 amino acids, such as 30 amino acids, thereby creating the third set of peptides comprising MHC class I binding peptides, MHC class II binding peptides and/or extended MHC class II binding peptides; or
ii. for each 15-mer peptide, determining the corresponding full-length variant protein sequence of step 1) with the highest sequence identity to the consensus sequence generated in step 2) and extending each 15-mer peptide with the determined corresponding full-length protein sequence that flanks the 15-mer peptide sequence to create one or more mosaic protein sequences, thereby creating the third set of peptides comprising MHC class I binding peptides, MHC class II binding peptides and/or mosaic protein sequences.

This extension step improves the likelihood that the longer peptides can emulate the 3-D structure of the native peptide hosting protein better. In some embodiments, this allows the peptides to elicit both the T-cell and B-cell response desirable for an efficient immune response for early clearance and/or protection against the target pathogen.

In some embodiments, step 10) comprises extending the 15-mer peptides that originate from proteins classified as at least partly extracellular using the consensus sequence generated for each protein in step 2) in the N- and C-terminal directions until the peptide length is between 25 to 35 amino acids, such as between 26 to 34 amino acids, such as between 27 to 33 amino acids, such as between 28 to 32 amino acids, such as between 29 to 31 amino acids, or such as 30 amino acids, thereby creating the third set of peptides comprising MHC class I binding peptides, MHC class II binding peptides and/or extended MHC class II binding peptides. Thus, if the extension in one of the C- or N-terminal directions reaches the end of the protein, the extension will continue in the other direction until the peptide sequence is the specified length.

Alternatively, step 10) may comprise extending each 15-mer peptide by first identifying the consensus sequence created in step 2) corresponding to the protein from which said 15-mer peptide originates, then identifying the full-length variant of the protein that has the highest sequence identity to the consensus sequence, and finally using the identified full-length protein variant as a template for extending the 15-mer peptide in the C- and N-terminal directions until the ends of the protein are reached. This results in a mosaic protein consisting of the 15-mer peptide flanked by amino acid sequences corresponding to the identified full-length protein variant.

Thus, in some embodiments, step 10) comprises extending the 15-mer peptides that originate from proteins classified as at least partly extracellular by determining the corresponding full-length variant protein sequence of step 1) with the highest sequence identity to the consensus sequence generated in step 2) and extending each 15-mer peptide with the determined corresponding full-length protein sequence that flanks the 15-mer peptide sequence to create one or more mosaic protein sequences, thereby creating the third set of peptides comprising MHC class I binding peptides, MHC class II binding peptides and/or mosaic protein sequences. In some embodiments, if two or more 15-mer peptide sequences are from the same protein, overlap, and are different in an epitope defining sequence, only one of the peptides is embedded in the mosaic protein sequence.

In some embodiments, the third set of peptides comprises or consists of peptide sequences each with a length between 8 to 35 amino acids, preferably between 9 and 30 amino acids, and optionally one or more full length mosaic proteins.

In some embodiments, the method as disclosed herein further comprises a step of in silico prediction of the 3-dimensional folding properties of one or more of the MHC class II binding peptides and/or extended MHC class II binding peptides in the third set of peptides. Said prediction may be performed by any useful method known to the skilled person in the art, e.g. such as those listed at https://en.wikipedia.org/wiki/List_of_disorder_prediction_software. In some embodiments, one or more of the MHC class II binding peptides and/or extended MHC class II binding peptides are scored for disorder (negative), structural uniqueness, and/or stability using a prediction algorithm. In some embodiments, said predication algorithm is Alphafold2. In some embodiments, said predication algorithm is proTstab.

The present methods are useful for generating peptide sets that can elicit immune responses directed towards a wide range of target pathogens.

In some embodiments, the target pathogen is selected from the group consisting of a bacteria, a fungus, a virus, a protozoa and a worm. In some embodiments, the target pathogen is a bacteria, such as Mycobacterium tuberculosis. In some embodiments, the target pathogen is a fungus, such as Candida auris. In some embodiments, the target pathogen is a virus, such as an influenza virus. In some embodiments, the target pathogen is a protozoa, such as Plasmodium falciparum. In some embodiments, the target pathogen is a worm, such as a trematode.

The present methods are additionally useful for generating peptide sets that can elicit immune responses directed towards a wide range of mutants or variants of a target pathogen.

In some embodiments, the number of said variants of said target pathogen is 5 or more. In some embodiments, the number of said variants of said target pathogen is as 10 or more. In some embodiments, the number of said variants of said target pathogen is 25 or more. In some embodiments, the number of said variants of said target pathogen is 50 or more. In some embodiments, the number of said variants of said target pathogen is 100 or more. In some embodiments, the number of said variants of said target pathogen is 150 or more. In some embodiments, the number of said variants of said target pathogen is 200 or more. In some embodiments, the number of said variants of said target pathogen is 250 or more. In some embodiments, the number of said variants of said target pathogen is 500 or more. In some embodiments, the number of said variants of said target pathogen is 1000 or more. In some embodiments, the number of said variants of said target pathogen is 2500 or more. In some embodiments, the number of said variants of said target pathogen is 5000 or more. In some embodiments, the number of said variants of said target pathogen is 10000 or more. In some embodiments, the number of said variants of said target pathogen is 50000 or more.

In some aspects of the present disclosure is provided a computer program product comprising instructions which, when the program is executed by a computer, cause the computer to carry out the method as described herein.

In some aspects is provided a computer-readable medium comprising instructions which, when executed by a computer, cause the computer to carry out the method as described herein.

In some aspects of the present disclosure is provided a set of propagated signals comprising computer readable instructions which, when executed by a computer, cause the computer to carry out the method as described herein.

In some aspects is provided a data processing system comprising a processor configured to perform the method as described herein.

### Compositions and Vaccines Comprising Peptide Sets Designed Using the Computer-Implemented Methods

The presently disclosed methods are useful for designing peptide sets for use in compositions, such as pharmaceutical compositions, e.g. vaccines.

In some aspects of the present disclosure is thus provided a composition comprising one or more peptides or one or more nucleic acids encoding said one or more peptides, wherein the one or more peptides are designed using to the method as described herein.

In some aspects is provided a pharmaceutical composition comprising one or more peptides or one or more nucleic acids encoding said one or more peptides, wherein the one or more peptides are designed using to the method as described herein, and a pharmaceutically acceptable diluent, carrier and/or excipient.

One or more peptides from the third set of peptides may directly be used for vaccine development. Alternatively, one or more peptides from the third set of peptides may be encoded as micro-genes in a DNA vaccine concept or as individual mRNAs in a mRNA vaccine concept. Some or all of the peptides from the third peptide set may also be intelligently fused into longer mRNAs or gene-like DNA constructs encoding poly-epitopes. Such a construct, with an optimized delivery system can create a broad and protective immune response.

In some aspects, the present disclose provides a method for producing and formulating a vaccine, said method comprising the steps of:
1) performing the method as disclosed herein; and
2) producing and formulating at least one peptide from the third set of peptides and/or a nucleic acid sequence encoding said peptide.

In some embodiments, the method as described herein above in the section “Computer-implemented methods for designing a set of peptides” thus further comprises producing and formulating at least one peptide from the third set of peptides for use in a vaccine. In some embodiments, the method as described herein above in the section “Computer-implemented methods for designing a set of peptides” thus further comprises producing and formulating a nucleic acid sequence encoding said peptide for use in a vaccine. In some embodiments, the method as described herein above in the section “Computer-implemented methods for designing a set of peptides” thus further comprises producing and formulating at least one peptide from the third set of peptides and one or more nucleic acid sequence encoding said peptide(s) for use in a vaccine.

In some embodiments, at least two peptides, such as at least 3 peptides, such as at least 5 peptides, such as at least 10 peptides, such as at least 15 peptides, such as at least 20 peptides, such as at least 25 peptides, such as at least 30 peptides, such as at least 40 peptides, such as at least 50 peptides, such as at least 75 peptides, such as at least 100 peptides, such as at least 125 peptides, such as at least 150 peptides, such as at least 175 peptides, or such as at least 200 peptides from the third set of peptides and/or the nucleic acid sequence encoding said peptides are formulated for use in a vaccine.

In some embodiments, the vaccine comprises at least one DNA polynucleotide encoding at least one peptide from the third set of peptides. In some embodiments, the vaccine comprises at least one mRNA polynucleotide encoding at least one peptide from the third set of peptides.

In some embodiments, the vaccine is a polyepitope vaccine.

In some embodiments, the vaccine comprises an mRNA or DNA polynucleotide encoding at least two peptides, such as at least 3 peptides, such as at least 4 peptides, such as at least 5 peptides, such as at least 10 peptides, such as at least 20 peptides, such as at least 30 peptides, such as at least 40 peptides, such as at least 50 peptides, such as at least 60 peptides, such as at least 70 peptides, such as at least 80 peptides, such as at least 90 peptides, such as at least 100 peptides, such as at least 125 peptides, such as at least 150 peptides, such as at least 175 peptides, or such as at least 200 peptides, from the third set of peptides. In some embodiments, two or more encoded peptides are separated by a linker. In some embodiments, each encoded peptide is separated by a linker. The skilled person will have no difficulty identifying and using appropriate linkers known in the art.

In some embodiments, the vaccine comprises at least two mRNA or DNA polynucleotides, such as at least 3 mRNA or DNA polynucleotides, such as at least 4 mRNA or DNA polynucleotides, such as at least 5 mRNA or DNA polynucleotides, such as at least 10 mRNA or DNA polynucleotides, such as at least 20 mRNA or DNA polynucleotides, such as at least 20 mRNA or DNA polynucleotides, such as at least 30 mRNA or DNA polynucleotides, such as at least 40 mRNA or DNA polynucleotides, such as at least 50 mRNA or DNA polynucleotides, such as at least 60 mRNA or DNA polynucleotides, such as at least 70 mRNA or DNA polynucleotides, such as at least 80 mRNA or DNA polynucleotides, such as at least 90 mRNA or DNA polynucleotides, such as at least 100 mRNA or DNA polynucleotides, such as at least 125 mRNA or DNA polynucleotides, such as at least 150 mRNA or DNA polynucleotides, such as at least 175 mRNA or DNA polynucleotides, or such as at least 200 mRNA or DNA polynucleotides, each encoding at least one peptide from the third set of peptides. In some embodiments, each mRNA or DNA polynucleotide only encodes a single peptide from the third set of peptides. In some embodiments, two or more mRNA or DNA polynucleotides are separated by a linker sequence. In some embodiments, each mRNA or DNA polynucleotide is separated by a linker sequence. In some embodiments, two or more encoded peptides are separated by a linker. In some embodiments, each encoded peptide is separated by a linker. The skilled person will have no difficulty identifying and using appropriate linkers and linker sequences known in the art.

In some embodiments, the polynucleotides are comprised within one or more vectors, such as one or more viral vectors or plasmids. In some embodiments, the viral vector is an adenoviral vector or a modified vaccinia Ankara (MVA) vector.

In some embodiments, said vaccine comprises at least one T cell epitope, such as a CD4⁺ T cell epitope or a CD8⁺ T cell epitope, or at least one B cell epitope. In some embodiments, said vaccine induces a humoral immune response or a cellular immune response.

The vaccine may comprise both peptides comprising T cell epitopes and peptides comprising B cell epitopes in order to stimulate a broad and protective immune response.

Thus, in some embodiments, said vaccine comprises at least one T cell epitope, such as a CD4⁺ T cell epitope or a CD8⁺ T cell epitope, and at least one B cell epitope. In some embodiments, said vaccine induces a cellular immune response and a humoral immune response.

In some embodiments, the vaccine comprises at least one CD4⁺ T cell epitope and at least one CD8⁺ T cell epitope.

### Uses and Methods of Treatment Comprising Peptide Sets Designed Using the Computer-Implemented Methods

The presently disclosed methods are useful for designing sets of peptides for use in a method of treatment.

In some aspects is provided the use of a peptide or a nucleic acid encoding said peptide, wherein the peptide is designed according to the methods as disclosed elsewhere herein, in the prophylaxis and/or treatment of a disease.

In some aspects is provided a peptide, or a nucleic acid encoding said peptide, designed according to the methods as disclosed herein for use in a method for treating and/or preventing a disease in a subject.

In some aspects is provided a method for treating and/or preventing a disease in a subject in need thereof, the method comprising administering to the subject a pharmaceutical composition as disclosed elsewhere herein.

In some embodiments, the pharmaceutical composition is administered to the subject once. In some embodiments, the pharmaceutical composition is administered to the subject twice over a period of time. In some embodiments, the pharmaceutical composition is administered to the subject three times over a period of time. In some embodiments, said period of time is 2 weeks, such as 3 weeks, such as 1 month, such as 2 months, such as 3 months, such as a 6 months, such as 9 months, such as 1 year, such as 1.5 years or such as 2 years.

In some embodiments, the subject is a mammal. In some embodiments, the mammal is a human.

### Kits of Parts

In some aspects of the present disclosure is provided a kit of parts comprising:
1) a composition or a pharmaceutical composition, such as a vaccine, as defined elsewhere herein; and
2) optionally, a medical instrument or other means for administering the composition; and
3) instructions for use.

### EXAMPLES

### Example 1—Designing a Candidate Peptide Set for Use in a Vaccine Against Influenza A

The present example relates to using the vaccine design process to design a set of peptides for use in a vaccine against Influenza A.

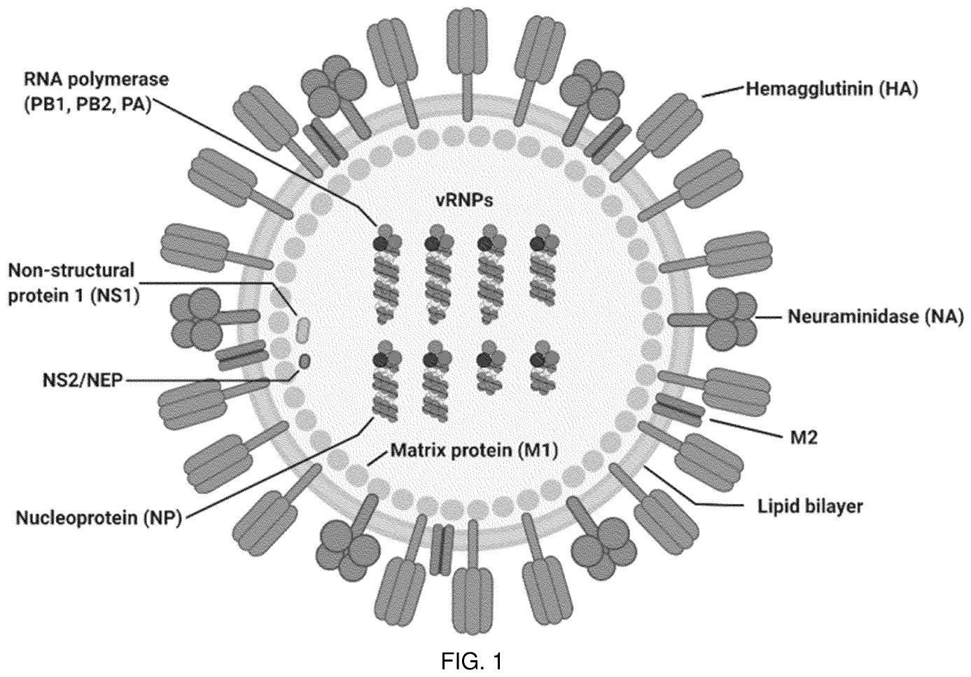

The process may comprise the following pre-processing steps:
1) Select the desired disease to create a vaccine against.
   Current example: Influenza A.
2) Define the pathogenic organism and range of variants causing the disease.
   Current example: Influenza A H1N1+H3N2.
3) Select pathogenic proteins important for establishing an infection (optional for smaller viral pathogens).
   Current example: All proteins.
4) Download protein sequences of all variants (incl. known mutants) of the pathogen strain(s) within the previously determined range.
   Current example: Download Influenza A H1N1+H3N2 strains with human as host from “NCBI Influenza Virus Resource.”
      i) Go to https://www.ncbi.nlm.nih.gov/genomes/FLU/Database/nph-select.cgi?go=database
      ii) In the part headed “Select sequence type:” select Protein
      iii) In the part headed “Define search set:”
         (1) In “Type” select A
         (2) In “Host” select Human
         (3) In “Country/Region” select any
         (4) In “Protein” select any
         (5) In “Subtype”+“H” select 1
         (6) In “Subtype”+“N” select 1
         (7) Leave “Sequence length”, “Collection date”, and “Release date” blank
         (8) Select “Full-length only”
         (9) Press button “Add query”
      iv) Repeat iii) except change (5) to [In “Subtype”+“H” select 3] and change (6) to [In “Subtype”+“H” select 1]
      v) Press button “Download Results”
5) Assign proteins as intracellular or extracellular
   a) Intracellular is here defined as the protein being expressed inside the infected cell, such as when the protein is not exported nor visible on the surface of the infected cell. Alternatively, a protein may be defined as intracellular even though it is exported or expressed on the surface of the cell, if it is known not to be important outside the cell for establishment or maintenance of an infection.
      Current example: Influenza A intracellular proteins: NP, M1, PA, PB1, PB2, NS1, NS2 (all proteins not defined as extracellular)
   b) Extracellular is here defined as a protein that is a cell/viral surface protein and is important for establishing or maintaining an infection.
      Current example: Influenza A extracellular proteins: HA, NA, M2 (M2e part) (See FIG. 1)
6) For each protein, assign how many peptides from the given protein there should be in the final selection.
   Current example: Three peptides from each extracellular protein and two peptides from each intracellular protein=23 peptides in total
7) Define target population, considering HLA loci, and the number of alleles for each locus.
   Current example:
      i) Target Population: Europe.
      ii) Considered HLA loci: HLA-A, HLA-B, HLA-C, and HLA-DRB1.
      iii) Number of alleles from each locus: Top 25 from each locus ranked decreasing by allele frequency.
   Alleles and the corresponding allele frequencies is formatted to fit input requirements for PopCover.

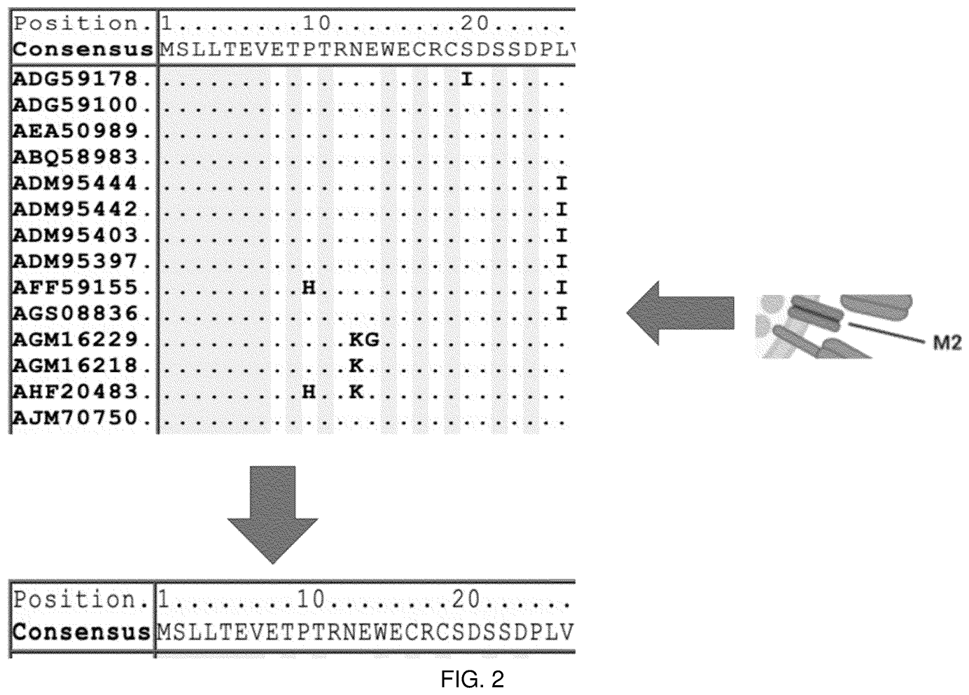

The pipeline may then comprise the following semi-automated steps:
1) All variant sequences of each protein assigned to be extracellular are aligned independently by a multiple alignment method, e.g., CLUSTALW, MAFFT or other method. For each protein, a consensus sequence is generated, e.g., using the most abundant amino acid at a given position.
   Current example: All downloaded variant sequences of each extracellular protein from the chosen Influenza A strains are aligned using the built-in multiple alignment tool from the download site to generate a multiple alignment and a consensus sequence. An example of the alignment and consensus sequence of the protein M2 is shown in FIG. 2.
2) Creation of digital sets of all possible unique 9-mers (9 AA long peptides) from all variant proteins for MHC class I binding prediction. Each 9mer is also stored with origin strain information for later processing to PopCover input.
3) Creation of digital sets of all possible unique 15-mers from all variant proteins for MHC class II binding prediction. Protein identity and peptide strain specificities will be assigned in parallel in a digital lookup table.
4) MHC class I predictions will be performed on all unique 9mer peptides, for all the selected HLA-A, HLA-B, and/or HLA-C alleles.
   Current Example: Predictions are performed by NetMHCpan version 4.1
5) MHC class II predictions will be performed on all unique 15-mer peptides, for all the selected HLA-DP, HLA-DQ, and/or HLA-DR alleles.
   Current Example: Predictions are performed by NetMHCIIpan version 4.0.
6) Predicted MHC binding peptides are fused with origin strain information and formatted for input to PopCover. Binding can be defined as an output score threshold, an affinity threshold, a rank threshold or any combination of these.
   Current Example: Peptides assigned as weak or strong binders by NetMHC are defined as binders.
7) (Optional) Only predicted binding peptides verified as having shown to give an immune response in experimental assays or database lookups are used in the below.
8) HLA binding peptides from each protein will be used as input for PopCover along with assigned HLA binding alleles and the corresponding allele frequencies in the relevant population. The resulting peptides with all relevant meta-data are stored in an independent table.
9) a) MHC class II binding peptides in the output of step 6 that originates from proteins assigned to be extracellular are extended with the consensus sequence of the aligned variant proteins in both directions until the peptide length is at least 30 AA. If the extension in one of the directions reaches the end of the protein the extension will continue in the opposite direction until the peptide sequence is 30 AA long
   OR
   b) Be encoded/formulated as individual peptides AND replace the original sequence in the protein variant closest to the consensus sequence to create a mosaic protein, and the full protein is encoded or used in the final construct/formulation. Note: In case the selected peptides from the same protein overlap and contradicts in the epitope defining sequence only one of the peptides will be embedded in the mosaic protein.
10) (Optional) Peptides from one or more peptide sets is validated for immunogenicity using in vivo assays, in vitro assays or database lookups. Then step 7 to 9 is repeated in order to find a high coverage peptide selection with validated immunogenic epitopes.

The above steps will result in a set of digital peptides of length 9AA-30AA and optionally one or more full length proteins. This set of peptides end protein(s) will, in the simplest form, directly be encoded as minigenes and optionally full protein gene(s) in a DNA vaccine concept or as individual mRNAs in a mRNA vaccine concept.

The peptide set might also be intelligently fused into longer mRNAs or gene-like DNA constructs encode so called poly-epitopes. Such a construct, with an optimized delivery system should be able to create a broad and protective immune response. However, some predicted HLA binding peptides may not be immunogenic and a number of cycles of experimental validation and new runs, leaving out known non-immunogenic peptides may be needed for a final optimal vaccine.

### Example 2—Validation of the Designed Peptide Sets

This example relates to various validation tests that may be used to validate pipeline according to the present disclosure, such as by validating the immunogenicity of the designed peptide sets for use in vaccines against a pathogen. The following example relates to vaccines against Influenza A.

### In Silico Tests

Validation will be performed by comparing peptide sets designed against circulating strains with vaccines already available against these strains, such as traditional vaccines against A(H1N1)pdm09 and A(H3N2):
1. Assess whether the number of predicted MHC class I epitopes designed using the pipeline selection method for A(H1N1)pdm09 or A(H3N2), using the chosen set of HLA alleles, are in A(H1N1)pdm09 or A(H3N2) traditional vaccines, respectively.
   a. For this assay, the predicted HLA coverage of the predicted epitopes of the peptide sets from the pipeline selection for A(H1N1)pdm09 or A(H3N2) may also be assessed.
2. Assess whether the number of predicted MHC class II epitopes for the surface protein subset of A(H1N1)pdm09 or A(H3N2) designed using the pipeline selection, using the chosen set of HLA alleles, are in A(H1N1)pdm or A(H3N2) traditional vaccines, respectively.
   a. For this assay, the predicted HLA coverage of the predicted surface protein epitopes of the peptide sets from the pipeline selection for A(H1N1)pdm09 or A(H3N2) may also be assessed.
3. Assess the number of verified HLA class I and HLA class II epitopes of peptide sets from the pipeline selection against A(H1N1)pdm09 or A(H3N2) that are also found in the Immune Epitope Database (IEDB)—https://www.iedb.org
   a. For this assay, the predicted HLA coverage of the verified epitopes may also be assessed.
   b. For this assay, the total HLA coverage of verified epitopes when considering only verified epitopes or epitopes with a measured HLA response may also be assessed.
4. Calculate the fraction of known H1N1 strains and known H3N2 strains that contain at least 3 verified HLA Class II epitopes and at least 5 verified HLA Class I epitopes designed according to the current method. A larger fraction of strain variants containing the given number of epitopes from the peptide set than from the traditional vaccines indicates the benefits of the computer implemented method described herein.

### In Vitro Tests

Similarly, peptide sets designed using the methods disclosed herein may be validated in vitro against commercially available vaccines.

Peptide sets selected using the process as disclosed herein will be produced as synthetic peptides by a number of commercial providers, e.g., Thermo Scientific Custom Peptide synthesis service from ThermoFisher.

These will be validated against commercially available vaccines, e.g., the 2020-2021 tetravalent influenza vaccine containing surface proteins from Influenza A H1N1 and Influenza A H3N2, or biologically produced proteins as defined by the vaccine sequences.

### Example 3—Designing a Set of Candidate Peptides for Use in a Vaccine Against Influenza A

The present example relates to using the vaccine design process to design a set of peptides for use in a vaccine against Influenza A.

### Pre-Processing Steps

The process may comprise the following pre-processing steps:
1) Select the desired disease to create a vaccine against.
   Current example: Influenza A
2) Define the pathogenic organism and variant range causing the disease.
   Current example: Influenza A H1N1+H3N2
3) Select pathogenic proteins important for establishing an infection (optional for smaller viral pathogens).
   Current example: All proteins
4) Download protein sequences of all variants (incl. known mutants) of the pathogen strain(s) within determined scope.
   Current example: Download Influenza A H1N1+H3N2 strains with human as host from “NCBI Influenza Virus Resource.”
   i) Go to https://www.ncbi.nlm.nih.gov/genomes/FLU/Database/nph-select.cgi?go=database
   ii) In the part headed “Select sequence type:” select Protein
   iii) In the part headed “Define search set:”
      (1) In “Type” select: A
      (2) In “Host” select: Human
      (3) In “Country/Region” select: any
      (4) In “Protein” select: any
      (5) In “Subtype”+“H” select: 1
      (6) In “Subtype”+“N” select: 1
      (7) Leave “Sequence length”, “Collection date”, and “Release date” blank
      (8) Select “Full-length only”
      (9) Press button “Add query”
   iv) Repeat iii) except change (5) to [In “Subtype”+“H” select 3] and change (6) to [In “Subtype”+“N” select 2]
   v) “Download Results”—Result set(CSV)
   vi) Fasta file and info xml file was also downloaded
   vii) 448343 influenza A sequences was downloaded

### Pipeline Steps

The pipeline may then comprise the following semi-automated steps:
1) Assign proteins as intracellular or extracellular
   a) For this example, an extracellular protein was defined as a protein that is a cell/viral surface protein and is important for establish/maintain an infection.
      Current example: Influenza A extracellular: HA and NA (See FIG. 1). M2(e) was not included because of its short length of only 30 amino acids.
   b) For this example, an intracellular protein was defined as being expressed inside an infected cell but will not be exported, nor be visible on the surface of the pathogen; or may be exported, or present on the surface of the pathogen but will not be important outside the cell for establish/maintain an infection).
      Current example: Influenza A intracellular: NP, M1, PA, PB1, PB2, NS1, NS2 (all proteins not defined as extracellular).
2) Define target population, considered HLA loci, and the number of alleles for each locus to consider
   Current example:
   i) Target Population: North Western Europe
   ii) A representative North Western European Population sample (e.g. a mixture of Swedish, Norwegian, Danish, English, Irish, German, Czech, Austrian, Belgian and Holland populations) is ideal to simplify later potential in vitro evaluation. However, large samples from at least three different countries within this selection should give a good approximation.
   iii) Considered HLA loci: HLA-A, HLA-B, HLA-C, and HLA-DRB1
   iv) From allelefrequencies.net the following available populations were selected:

| | |
|---|---|
| (1) | Germany pop 6 (pop_id=2752, 8862 subjects, A,B,C and DRB1), |
| | Netherlands Leiden (pop_id=3257, 1305 subjects, A,B,C, DRB1, |
| | DQB1), Czech Republic NMDR, (pop_id=3258, 5099 subjects, |
| | A,B,C, DRB1 and DQB1), Northern Ireland (pop_id=1243, 1000 |
| | subjects, A, B, C, DRB1, DQB1) |

v) The average top 10 alleles in Europe for each locus as defined by allelefrequencies.net as of 17 May 2022 were used.
   (1) HLA-A*01:01, HLA-A*02:01, HLA-A*03:01, HLA-A*11:01, HLA-A*24:02, HLA-A*26:01, HLA-A*29:02, HLA-A*31:01, HLA-A*32:01, and HLA-A*68:01
   (2) HLA-B*07:02, HLA-B*08:01, HLA-B*15:01, HLA-B*18:01, HLA-B*27:05, HLA-B*35:01, HLA-B*40:01, HLA-B*44:02, HLA-B*44:03, and HLA-B*51:01
   (3) HLA-C*02:02, HLA-C*02:09, HLA-C*03:03, HLA-C*03:04, HLA-C*04:01, HLA-C*05:01, HLA-C*06:02, HLA-C*07:01, HLA-C*07:02, and HLA-C*12:03
   (4) HLA-DRB1*01:01, HLA-DRB1*03:01, HLA-DRB1*04:01, HLA-DRB1*07:01, HLA-DRB1*11:01, HLA-DRB1*11:04, HLA-DRB1*13:01, HLA-DRB1*13:02, HLA-DRB1*15:01, and HLA-DRB1*16:01
vi) The selected alleles are calculated to cover the population as follows (how big a fraction of the given population have at least one of the selected alleles present). The overall frequency for each allele (fa) is calculated as follows where fₐₚ is frequency of the given allele in sub-population p and Sp is the sample size of sub-population p:

$$fa = \frac{{\sum }_{p ∈ populations} {f}_{ap} \times {S}_{p}}{{\sum }_{p ∈ populations} {S}_{p}}$$

(1) HLA-A locus: 99%
(2) HLA-B locus: 91%
(3) HLA-C locus: 97%
(4) HLA-DRB1 locus: 96%
3) All protein sequences from the downloaded Influenza A sequences were compiled to all 9-mer peptides present within any protein sequence considered to be an intracellular protein (sub-9-mer peptides).
   a) Only one instance of each sub-peptide is kept but information about the specific pathogen and protein origins are linked to the peptide sequence. 205626 unique 9-mer peptides were compiled.
   b) MHC binding were predicted for all unique 9-mer peptides to the 30 HLA-A, HLA-B, and HLA-C alleles using NetMHCpan-4.1.
   c) Default settings used:
      NetMHCpan-4.1 binder (strong or weak) rank below 2.0%
   d) Only the 9-mer peptides predicted to be strong or weak binders to one or more of the above selected HLA-alleles were considered. 72629 unique 9mer peptides were saved together with the information of which alleles were predicted to bind the peptide as well as the information regarding strain and protein origins of the peptide.
4) Protein sequences from the downloaded Influenza A sequences belonging to the assigned external proteins are compiled to all 15-mer peptides present within any protein sequence (sub-15-mer peptides).
   a) Only one instance of each sub-peptide is kept but information about the specific pathogen and protein origins were linked to each sub-peptide sequence. 380304 unique 15-mer peptides were compiled.
   b) MHC binding were predicted for all unique 15-mer sub-peptides to the 10 HLA-DRB1 alleles using NetMHCIIpan-4.1
   c) Default settings used:
      NetMHCIIpan-4.1 binder (strong or weak) rank below 5.0%
   d) Only the 15-mer peptides predicted to be either weak or strong binders to one or more of the above selected HLA-alleles were considered. 99370 15-mer peptides were saved together with the information of which alleles that were predicted to bind the peptide, the predicted 9-mer binding core, as well as the information regarding strain and protein origins of the peptide.
5) Assign how many peptides there should be in the intermediate selection.
   Current example: Eighty (80) peptides from extracellular proteins and eighty (80) peptides from intracellular proteins.
6) An implementation of an algorithm that optimizes population coverage of a selected number of peptides given the above-described inputs is shown in the section below (“Example algorithm”).
7) The algorithm defined in point 6) was run 10 times with inputs of predicted HLA class I binding 9-mer peptides, HLAs binding each peptide, and population coverage of the given HLA allele and resulting in sets of eight (8) peptides for each run. After each run the predicted 8-peptide set was removed from the total pool of predicted binding 9-mers. This resulted in 8×10=80 9-mer peptides taken forward and designated set I.1
8) The algorithm from point 6) was run 10 times with inputs of predicted binding 15-mer peptides, HLAs binding each peptide, and population coverage of the given HLA allele and resulting in sets of eight (8) peptides for each run. After each run the predicted 8-peptide set was removed from the total pool of predicted binding 15mers. This resulted in 8×10=80 15-mer peptides taken forward and designated set 11.1.
9) As a proxy for experimental validation of actual immunogenicity of selected predicted binders the database FluDB, from which it is possible to download validated Influenza epitopes, was used (https://www.fludb.org/brc/influenza_epitope_search.spg?method=ShowCleanSearch&decorator=influenza).
10) All available Human Influenza A epitopes validated to have given a positive T-Cell response were downloaded.
11) The epitopes downloaded in point 10 were cleaned to ensure that only Human host and positive T-cell Assays epitopes were considered forward.
   a) Positive class I epitopes: 205
   b) Positive class II epitopes: 443
   As some assays are performed using extended peptides that will be digested to smaller peptides under the assay conditions, all peptides from set I.1 that were found identical as a full sub-peptide within a validated epitope were considered as positive responding peptides (Kozlowski et al., 1993)
12)
   a) It was assumed that the peptides of set I.1 deemed as positive are positive for any of the alleles predicted to bind the given peptide, disregarding the serotype of the donor to have delivered the test cells.
   b) Number of set I.1 peptides found in positive epitope set: 20
   c) The 20 positive set I.1 peptides was named set I.2
13) IMHC class II presented peptides originate from extended peptides, or from full proteins that have been digested to smaller peptides during the process resulting in loading and final presentation. For this reason, each 15-mer peptide from set II.1 with a predicted 9-mer binding core found as an identical full sub-peptide within a validated epitope was considered to be a positive responding peptide.
   a) It is assumed that the peptides of set II.1 deemed as positive are positive for any of the alleles predicted to bind the given peptide, disregarding the serotype of the donor to have delivered the test cells.
   b) Number of set II.1 peptides found in positive epitope set: 61
   c) The 61 positive set II.1 peptides was named set II.2
14) The algorithm from point 6) was performed with set I.2 as input set to deliver a set of eight (8) peptides covering an optimal fraction of the considered population.
   a) The resulting peptide set (set I.3) composition and coverage is displayed in Table 1-3, below. Total Coverage is calculated as the product of HLA coverage and Variant coverage. Overall is the cumulative coverage.

TABLE 1

| | HLA-A | HLA-B | HLA-C | HLA coverage |
|---|---|---|---|---|
| YQKRMGVQ | 0.0% | 47.5% | 87.4% | 93.4% |
| M | | | | |
| FMYSDFHFI | 66.6% | 10.2% | 97.1% | 99.1% |
| GINDRNFWR | 45.6% | 0.0% | 0.0% | 45.6% |
| TQIQTRRSF | 29.3% | 70.1% | 87.4% | 97.3% |
| ITFMQALQL | 6.8% | 10.2% | 74.7% | 78.8% |
| SLLTEVETY | 44.4% | 32.5% | 36.1% | 76.0% |
| KTRPILSPL | 11.2% | 25.5% | 63.7% | 76.0% |
| YRYGFVANF | 27.4% | 6.9% | 86.4% | 90.8% |
| Overall | 98.6% | 89.6% | 97.1% | 100.0% |

TABLE 2

| | | | | | | | | Variant |
|---|---|---|---|---|---|---|---|---|
| | M1 | NP | NS1 | NS2 | PA | PB1 | PB2 | coverage |
| YQKR | 99.8% | 0.0% | 0.0% | 0.0% | 0.0% | 0.0% | 0.0% | 99.8% |
| MGVQ | | | | | | | | |
| M | | | | | | | | |
| FMYSD | 0.0% | 0.0% | 0.0% | 0.0% | 99.8% | 0.0% | 0.0% | 99.8% |
| FHFI | | | | | | | | |
| GINDR | 0.0% | 99.8% | 0.0% | 0.0% | 0.0% | 0.0% | 0.0% | 99.8% |
| NFWR | | | | | | | | |
| TQIQT | 0.0% | 0.0% | 0.0% | 0.0% | 0.0% | 99.3% | 0.0% | 99.3% |
| RRSF | | | | | | | | |
| ITFMQ | 0.0% | 0.0% | 0.0% | 99.7% | 0.0% | 0.0% | 0.0% | 99.7% |
| ALQL | | | | | | | | |
| SLLTE | 99.8% | 0.0% | 0.0% | 0.0% | 0.0% | 0.0% | 0.0% | 99.8% |
| VETY | | | | | | | | |
| KTRPI | 100.0% | 0.0% | 0.0% | 0.0% | 0.0% | 0.0% | 0.0% | 100.0% |
| LSPL | | | | | | | | |
| YRYGF | 0.0% | 0.0% | 0.0% | 0.0% | 0.0% | 99.8% | 0.0% | 99.8% |
| VANF | | | | | | | | |
| Overall | 100.0% | 99.8% | 0.0% | 99.7% | 99.8% | 100.0% | 0.0% | 100.0% |

TABLE 3

| | HLA | Variant | Total |
|---|---|---|---|
| | coverage | coverage | Coverage |
| YQKRMGVQM | 93.4% | 99.8% | 93.2% |
| FMYSDFHFI | 99.1% | 99.8% | 98.9% |
| GINDRNFWR | 45.6% | 99.8% | 45.5% |
| TQIQTRRSF | 97.3% | 99.3% | 96.6% |
| ITFMQALQL | 78.8% | 99.7% | 78.6% |
| SLLTEVETY | 76.0% | 99.8% | 75.8% |
| KTRPILSPL | 76.0% | 100.0% | 76.0% |
| YRYGFVANF | 90.8% | 99.8% | 90.6% |
| Overall | 100.0% | 100.0% | 100.0% |

15) The algorithm from point 6) was used with set II.2 as input set to deliver a set of eight (8) peptides covering an optimal fraction of the considered population.
   a) The resulting peptide set (set II.3) coverage is displayed in Table 4, below.

TABLE 4

| | HLA | | | | |
|---|---|---|---|---|---|
| | (DRB1) | | | Variant | Total |
| | coverage | NA | HA | Coverage | coverage |
| VPDYASLRSLVASSG | 63.2% | 0.0% | 60.3% | 60.3% | 38.1% |
| TDTIKSWRNNILRTQ | 64.9% | 36.9% | 0.0% | 36.9% | 23.9% |
| FAPFSKDNSIRLSAG | 68.3% | 54.7% | 0.0% | 54.7% | 37.4% |
| GDKITFEATGNLVVP | 71.5% | 0.0% | 31.0% | 31.0% | 22.2% |
| AASYKIFKIEKGKVT | 89.8% | 4.3% | 0.0% | 4.3% | 3.9% |
| VKSQLKNNAKEIGNG | 54.9% | 0.0% | 4.2% | 4.2% | 2.3% |
| SYKIFRIEKGKIIKS | 84.3% | 16.4% | 0.0% | 16.4% | 13.8% |
| NAELLVALENQHTID | 56.0% | 0.0% | 62.1% | 62.1% | 34.8% |
| Cumulative | 95.8% | 97.7% | 98.3% | 100.0% | 95.8% |
| coverage | | | | | |

16) All variant sequences belonging to each of the 2 proteins classified as external proteins were saved in 2 individual files in FASTA format and a multiple alignment was created for each protein using MAFFT v7.149b (2014/04/04) with the following option: “--legacygappenalty”.
17) Consensus sequences were made using the multiple alignment file created in point 16). The most common amino acid at each position in the sequence was assigned as the amino acid in the consensus sequence. In cases where 50% or more of the aligned variant sequences showed a gap assigned to a position, said position was disregarded and skipped.
   a) Resulting consensus sequences:

i) Hemagglutinin (HA) HA_consensus
(SEQ ID NO: 2)
MKTIIALSYILCLVFAQKIPGNDNSTATLCLGHHAVPNGTIVKTI
TNDRIEVTNATELVQNSSIGEICDSPHQILDGENCTLIDALLGDP
QCDGFQNKKWDLFVERSKAYSNCYPYDVPDYASLRSLVASSGTLE
FNNESFNWTGVTQNGTSSACIRASASSFFSRLNWLTHLNYSYPAL
NVTMPNNEQFDKLYIWGVHHPGTDKDQIFLYAQSSGRITVSTKRS
QQAVIPNIGSRPRVRDIPSRISIYWTIVKPGDILLINSTGNLIA
PRGYFKIRSGKSSIMRSDAPIGKCKSECITPNGSIPNDKPFQNVN
RITYGACPRYVKQSTLKLATGMRNVPEKQTRGIFGAIAGFIENGW
EGMVDGWYGFRHQNSEGRGQAADLKSTQAAIDQINGKLNRLIGKT
NEKFHQIEKEFSEVEGRIQDLEKYVEDTKIDLWSYNAELLVALEN
QHTIDLTDSEMNKLFEKTKKQLRENAEDMGNGCFKIYHKCDNACI
GSIRNGTYDHNVYRDEALNNRFQIKGVELKSGYKDWILWISFAIS
CFLLCVALLGFIMWACQKGNIRCNICI.
ii) Neuraminidase (NA)-NA_consensus
(SEQ ID NO: 3)
MNPNQKIITIGSVSLTISTICFFMQIAILITTVTLHFKQYEFNSP
PNNQVMLCEPTIIERNITEIVYLTNTTIEKEICPKLAEYRNWSKP
QCPITGFAPFSKDNSIRLSAGGDIWVTREPYVSCDPDKCYQFALG
QGTTLNNVHSNNTVRDRTPYRTLLMNELGVPFHLGTKQVCIAWSS
SSCHDGKAWLHVCITGDDKNATASFIYNGRLVDSIVSWSKNILRT
QESECVCINGTCTVVMTDGPADGKADTKILFIEEGKIVHTSELSG
SAQHVEECSCYPRYPGVRCVCRDNWKGSNRPIVDINIKDYSIVSS
YVCSGLVGDTPRKNDSSSSSHCLGPNNEEGGHGVKGWAFDDGNDV
WMGRTISETSRLGYETFKVPEGWSNTKSKLQINRQVIVDRGDRSG
YSGIFSVEGKSCINRCFYVELIRGRKEETEVLWTSNSIVVFCGTS
GTYGTGSWPDGADLNLMII.

18) The closest match of each of the 15mer peptides to the consensus sequences was identified using BLAST SWIPE 2.0.5 [Aug. 9 2012 11:48:15].
   a) HA peptides elongated 7 amino acids towards the N terminal and 8 amino acids towards the C terminal:

(SEQ ID NO: 4)
VPDYASLRSLVASSG =>
(SEQ ID NO: 5)
SNCYPYDVPDYASLRSLVASSGTLEFNNES
(SEQ ID NO: 6)
GDKITFEATGNLVVP =>
(SEQ ID NO: 7)
YWTIVKPGDKITFEATGNLVVPRGYFKIRS
(SEQ ID NO: 8)
VKSQLKNNAKEIGNG =>
(SEQ ID NO: 9)
MNKLFEKVKSQLKNNAKEIGNGCFKIYHKC
(SEQ ID NO: 10)
NAELLVALENQHTID =>
(SEQ ID NO: 11)
KIDLWSYNAELLVALENQHTIDLTDSEMNK

b) NA peptides elongated 7 amino acids towards the N terminal and 8 amino acids towards the C terminal:

(SEQ ID NO: 12)
TDTIKSWRNNILRTQ =>
(SEQ ID NO: 13)
FIYNGRLTDTIKSWRNNILRTQESECVCIN
(SEQ ID NO: 14)
FAPFSKDNSIRLSAG =>
(SEQ ID NO: 15)
PQCPITGFAPFSKDNSIRLSAGGDIWVTRE
(SEQ ID NO: 16)
AASYKIFKIEKGKVT =>
(SEQ ID NO: 17)
TDGPADGAASYKIFKIEKGKVTHTSELSGS
(SEQ ID NO: 18)
SYKIFRIEKGKIIKS =>
(SEQ ID NO: 19)
GPADGKASYKIFRIEKGKIIKSSELSGSAQ

19) Final peptide cocktail suggested for inclusion in a vaccine for protection against all variants of influenza A H1N1 and H3N2 optimized for eliciting an immune response in the North Western European population:

(SEQ ID NO: 20)
YQKRMGVQM
(SEQ ID NO: 21)
FMYSDFHFI
(SEQ ID NO: 22)
GINDRNFWR
(SEQ ID NO: 23)
TQIQTRRSF
(SEQ ID NO: 24)
ITFMQALQL
(SEQ ID NO: 25)
SLLTEVETY
(SEQ ID NO: 26)
KTRPILSPL
(SEQ ID NO: 27)
YRYGFVANF
(SEQ ID NO: 5)
SNCYPYDVPDYASLRSLVASSGTLEFNNES
(SEQ ID NO: 7)
YWTIVKPGDKITFEATGNLVVPRGYFKIRS
(SEQ ID NO: 9)
MNKLFEKVKSQLKNNAKEIGNGCFKIYHKC
(SEQ ID NO: 11)
KIDLWSYNAELLVALENQHTIDLTDSEMNK
(SEQ ID NO: 13)
FIYNGRLTDTIKSWRNNILRTQESECVCIN
(SEQ ID NO: 15)
PQCPITGFAPFSKDNSIRLSAGGDIWVTRE
(SEQ ID NO: 17)
TDGPADGAASYKIFKIEKGKVTHTSELSGS
(SEQ ID NO: 19)
GPADGKASYKIFRIEKGKIIKSSELSGSAQ

### Example Algorithm

This is a remake of the PopCover suitable for influenza.

Here also all proteins (corresponding to the RNA segments that in principle can be reshuffled independently between strains) are sought to get covered.

Scoring aims to always be a number between 0 and 1.

### Definitions Regarding HLA Coverage

L∈{A,B,C,DRB1}

H_{A}∈{*01:01,*02:01,*03:01,*11:01,*24:02,*26:01,*29:02,*31:01,*32:01,*68:01}

H_{B}∈{*07:02,*08:01,*15:01,*18:01,*27:05,*35:01,*40:01,*44:02,*44:03,*51:01}

H_{C}∈{*02:02,*02:09,*03:03,*03:04,*04:01,*05:01,*06:02,*07:01,*07:02,*12:03}

H_{DRB1}∈{*01:01,*03:01,*04:01,*07:01,*11:01,*11:04,*13:01,*13:02,*15:01,*16:01}

HLA frequency from DB:

f_{H}_{_{L}}

Number of peptides in the current selection that is predicted to bind to a given HLA:

n_{H}_{_{L}}

Penalty factor to reduce the impact of already covered HLAs:

β=10

### Definitions Regarding Genome Coverage:

Intracellular protein types:

P_{I}∈{M1,NP,NS,NS2,PA,PB1,PB2}

Extracellular protein types:

P_{E}∈{HA,NA}

All protein types:

P_{IE}∈{P_{I}∪P_{E}}

All protein variants:

p∈{all variants of all proteins}

Number of peptides in the current selection that is a genuine sub-peptide of a given protein sequence (i.e., how many times is this specific protein targeted by the current peptide selection):

nₚ

Total number of proteins:

np

Fraction of all protein variants a given protein variant accounts for

$${f}_{p} = \frac{1}{np}$$

### Scoring Calculation

### HLA Part

Before selecting the next peptide the scores will be calculated:

$$∀ {H}_{L} ; {n}_{{H}_{L}} > 0.$$

$${S}_{{H}_{L}} = \frac{{f}_{{H}_{L}}}{{β}^{{n}_{{H}_{L}}}}$$

$${R}_{L} = \sum {f}_{{H}_{L}} - {S}_{{H}_{L}}$$

$$∀ {H}_{L} ; {n}_{{H}_{L}} = 0.$$

$${S}_{{H}_{L}} = \frac{{f}_{{H}_{L}}}{1 - {R}_{L}}$$

$$\begin{matrix}Genome Part & \\ ∀ p ; {n}_{p} > 0. : {S}_{p} = \frac{{f}_{p}}{{β}^{{n}_{p}}} & \end{matrix}$$

$${R}_{p} = \sum {f}_{p} - {S}_{p}$$

$$∀ p ; {n}_{p} = 0. : {S}_{p} = \frac{{f}_{p}}{1 - {R}_{p}}$$

### Final Score

The final peptide score used in each iteration is the product of the genome score and the HLA score:

$$S = {S}_{p} \times {S}_{{H}_{L}}$$

In each iteration the peptide with the largest S is selected.

### Example 4—Validation of the Designed Peptide Sets from Example 3

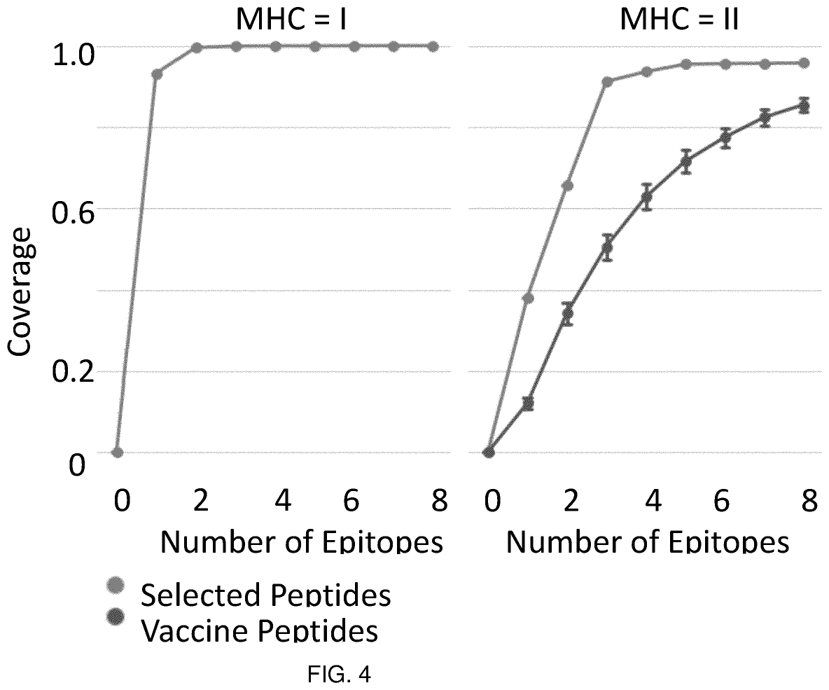

1) To validate the selected minimal peptide based selection against the currently used vaccination approach, population coverage of the currently used vaccine strains was validated.
   Using information from SSI (https://en.ssi.dk/surveillance-and-preparedness/surveillance-in-denmark/annual-reports-on-disease-incidence/influenza-season-2017-2018) about the recommended vaccine for 2018/2019, the following H1N1 and H3N2 strains should be used:
      i) A/Michigan/45/2015 (H1N1) pdm09-like virus
      ii) A/SingaporeINFIMH-16-0019/2016 (H3N2)-like virus (NEW VIRUS)
   However, the Singapore strain was not identified in the SSI Database 2018/2019, thus the information from SSI about the recommended vaccine for 2019/2020 (Influenza season 2018/19, The influenza season—report on disease occurrence in 2018/19, Updated 26 Jun. 2019, which is incorporated herein) could be used. Thus, according to said database the following H1N1 and H3N2 strains should be used:
      i) A/Brisbane/02/2018 (H1N1)pdm09-like virus (NEW VIRUS)
      ii) A/Kansas/14/2017 (H3N2)-like virus (NEW VIRUS)
   The Brisbane strain could not be identified in the Database, thus for validation it was used the following combination:
      i) A/Michigan/45/2015 (H1N1) pdm09-like virus
      ii) A/Kansas/14/2017 (H3N2)-like virus (NEW VIRUS)
2) Sub-9-mers hosted in the two strains and which were included in the 205 positive class I epitopes defined in step 14 a) of Example 3 were defined as “MHC I Vaccine set”. The set contains 180 9-mer peptides which are all predicted to bind to one of the 30 selected HLA-A, -B or -C alleles.
3) Sub-15-mers hosted in the two strains and included in the 443 positive class II epitopes defined in step 15 a) of Example 3 were defined as “MHC II Vaccine set”. The set contains 216 15-mer peptides, which are all predicted to bind to one of the 10 selected DRB1 alleles.
4) Validation goal
   To show that in general maximal coverage is reached for MHC Class I selected peptides, and that maximal coverage can be reached with significantly fewer epitopes for MHC Class II selected peptides with the defined selection procedure compared to taking a random equally sized subset of the validated epitopes. Maximal theoretical coverage for MHC I epitopes is 100%. Maximal theoretical coverage for MHC II epitopes is 96% (step 2 section a)_vi_(4))
5) Validation results
   The validation goal was reached as can be seen in FIG. 4. As can be seen in the figure, maximal coverage for MHC class I epitopes is reached with only 2 peptide epitopes in the set. Maximal coverage is reached for MHC class II epitopes with significantly fewer peptide epitopes in sets designed according to the presently disclosed methods compared to sets consisting of randomly selected validated vaccine epitopes.

### CONCLUSION

The inventors have shown that with the method of the present disclosure it is possible to select a small set of distinct peptides that are suitable in stimulating three legs of the adaptive immune system (cytotoxic T cells, helper T cells, and antibody producing B cells), thus securing good immunological protection and ensuring maximal population and variant coverage over all Influenza A H1N1 variants and Influenza A H3N2 variants. This is in contrast to the vaccines used today, which are inactivated strain specific vaccines. These generate only humoral immunity (Keshavarz et al., 2019), and the protective response is known to only be specific to the strains used to generate the vaccine (Nypaver et al., 2021).

### Items

1. A computer implemented method for designing a set of peptides, the method comprising the steps of:
   1) providing a computer-readable list of protein sequences encoded by a target pathogen genome, wherein
      i. the list further comprises protein sequences encoded by a genome of at least one variant of said target pathogen (variant protein sequences); and
      ii. each protein sequence from a protein that is at least partly extracellular is assigned a computer-readable classifier;
   2) aligning said variant protein sequences for each at least partly extracellular protein sequence by multiple alignment and generating a consensus sequence for each extracellular protein;
   3) optionally, creating an 8-11-mer peptide set comprising all unique 8-, 9-, 10- and/or 11-mer peptide sequences for all protein sequences;
   4) optionally, creating a 15-mer peptide set comprising all unique 15-mer peptide sequences for all protein sequences;
   5) predicting MHC class I binding for each unique 8-11-mer peptide, for at least one human leukocyte antigen (HLA) allele selected from the group consisting of HLA-A, HLA-B and HLA-C;
   6) predicting MHC class II binding for each unique 15-mer peptide, for at least one HLA allele selected from the group consisting of HLA-DP, HLA-DQ and HLA-DR;
   7) creating a first set of selected peptides, wherein the first set of selected peptides comprises 8-11-mer and/or 15-mer peptides that are predicted to bind to at least one HLA allele with a minimum binding score;
   8) optionally, validating the immunogenicity of one or more peptides from the first set of peptides, such as by an in vivo assay, an in vitro assay and/or by database lookup, thereby generating a first set of validated peptides,
   9) combining
      i. the first set of selected peptides;
      ii. the corresponding HLA alleles predicted to bind each peptide in the first set of selected peptides; and
      iii. HLA allele frequencies in a human target population,
      or
      i. the first set of validated peptides;
      ii. the corresponding HLA alleles predicted to bind each peptide in the first set of validated peptides; and
      iii. HLA allele frequencies in a human target population,
      to generate a second set of selected peptides, wherein the second set of peptides comprises peptides that, when taken together, are
      i. present in at least 75%, such as at least 80%, such as at least 85%, such as at least 90%, or such as at least 95% of said variants of said target pathogen; and
      ii. predicted to be bound by at least one HLA allele present in said human target population, in at least 75%, such as at least 80%, such as at least 85%, such as at least 90%, or such as in at least 95% of said human target population;
   10) creating a third set of peptides from the second set of selected peptides by
      i. extending the 15-mer peptides that originate from proteins classified as at least partly extracellular using the consensus sequence generated for each protein in step 2) in the N- and C-terminal directions until the peptide length is between 25 to 35 amino acids, such as between 28 to 32 amino acids, such as 30 amino acids, thereby creating the third set of peptides comprising MHC class I binding peptides, MHC class II binding peptides and/or extended MHC class II binding peptides; or
      ii. for each 15-mer peptide, determining the corresponding full-length variant protein sequence of step 1) with the highest sequence identity to the consensus sequence generated in step 2) and extending each 15-mer peptide with the determined corresponding full-length protein sequence that flanks the 15-mer peptide sequence to create one or more mosaic protein sequences, thereby creating the third set of peptides comprising MHC class I binding peptides, MHC class II binding peptides and/or mosaic protein sequences.
2. The method according to item 1, further comprising a step 11) of validating the immunogenicity of one or more peptides from the third set of peptides, such as by an in vivo assay, an in vitro assay and/or by database lookup, thereby generating a second set of validated peptides, and optionally repeating steps 8) to 9) using said second set of validated peptides.
3. The method according to any one of the preceding items, wherein protein sequences from a protein that is at least partly extracellular and which is known to not be important outside the cell for establishment or maintenance of an infection are removed from said computer-readable list provided in step 1).
4. The method according to any one of the preceding items, wherein
   i. predicting MHC class I binding for each unique 8-11-mer peptide in step 5) is performed using an algorithm selected from the list consisting of NetMHCpan, MHCSeqNet, NetMHC, NetMHCcons, PickPocket and MHCflurry, preferably wherein predicting MHC class I binding for each unique 8-11-mer peptide in step 5) is performed using NetMHCpan; and/or
   ii. predicting MHC class II binding for each unique 15-mer peptide in step 6) is performed using an algorithm selected from the list consisting of NetMHCIIpan, MARIA and MoDec, preferably wherein predicting MHC class II binding for each unique 15-mer peptide in step 6) is performed using NetMHCIIpan.
5. The method according to any one of the preceding items, wherein the second set of selected peptides of step 9) is generated using the PopCover algorithm.
6. The method according to any one of the preceding items, wherein
   i. if the extension of the 15-mer peptide of step 10) i. in the C- or N-terminal direction reaches the end of the corresponding protein consensus sequence, the extension is continued at the opposite terminal until the peptide length is between 25 to 35 amino acids, such as between 28 to 32 amino acids, preferably the extension is continued at the opposite terminal until the peptide length is 30 amino acids; or
   ii. if two or more 15-mer peptide sequences of step 10) ii. are from the same protein, overlap, and are different in an epitope defining sequence, only one of the peptides is embedded in the mosaic protein sequence.
7. The method according to any one of the preceding items, wherein if two or more 15-mer peptide sequences of step 10) ii. are from the same protein, overlap, and are different in an epitope defining sequence, only one of the peptides is embedded in the mosaic protein sequence.
8. The method according to any of the preceding items, further comprising producing and formulating at least one peptide from the third set of peptides and/or a nucleic acid sequence encoding said peptide for use in a vaccine.
9. The method according to item 8, wherein said vaccine comprises at least one T cell epitope, such as CD4⁺ T cell epitope, and/or at least one B cell epitope.
10. The method according to any one of items 8 to 9, wherein said vaccine induces a cellular immune response and/or a humoral immune response.
11. The method according to any one of items 8 to 10, wherein the vaccine comprises an mRNA or DNA polynucleotide encoding at least one peptide, such as at least two peptides, such as at least 3 peptides, such as at least 4 peptides, such as at least 5 peptides, such as at least 10 peptides, or such as at least 20 peptides from the third set of peptides.
12. The method according to any one of items 8 to 11, wherein the vaccine is a polyepitope vaccine.
13. A computer-readable medium comprising instructions which, when executed by a computer, cause the computer to carry out the method according to any one of items 1 to 12.
14. A composition comprising one or more peptides or one or more nucleic acids encoding said one or more peptides, wherein the one or more peptides are designed using to the method according to any one of items 1 to 12.
15. A peptide, or a nucleic acid encoding said peptide, wherein the peptide is designed according to the method of any one of items 1 to 12, for use in a method for treating and/or preventing a disease in a subject.

1. A computer implemented method for designing a set of peptides, the method comprising the steps of:
a) providing a computer-readable list of protein sequences encoded by a target pathogen genome, wherein
i. the list further comprises protein sequences encoded by a genome of at least one variant of said target pathogen (“variant protein sequences”); and
ii. each protein sequence from a protein that is at least partly extracellular is assigned a computer-readable classifier;
b) aligning said variant protein sequences for each at least partly extracellular protein sequence by multiple alignment and generating a consensus sequence for each extracellular protein;
c) creating a 15-mer peptide set comprising all unique 15-mer peptide sequences for all protein sequences;
d) determining MHC class II binding for each unique 15-mer peptide, for at least one MHC class II allele, such as for at least one HLA allele selected from the group consisting of HLA-DP, HLA-DQ and HLA-DR;
e) creating a first set of selected peptides, wherein the first set of selected peptides comprises the unique 15-mer peptides that are predicted to bind to at least one MHC class II allele, such as at least one HLA allele, with a minimum binding score;
f) i. validating the immunogenicity of one or more peptides from the first set of peptides, such as by at least one of an in vivo assay, an in vitro assay, by database lookup, thereby generating a first set of validated peptides, and combining data describing the first set of validated peptides, the corresponding MHC class II alleles predicted to bind each peptide in the first set of validated peptides, and MHC class II allele frequencies in a target population; or
ii. combining data describing the first set of selected peptides, the corresponding MHC class II alleles predicted to bind each peptide in the first set of selected peptides, and MHC class II allele frequencies in a target population;
g) generating a second set of selected peptides, wherein the second set of peptides comprises peptides that, when taken together, are
i. present in at least 75 of said variants of said target pathogen; and
ii. predicted to be bound by at least one MHC class II allele present in said target population, in at least 75 of said target population;
h) creating a third set of peptides from the second set of selected peptides by
i. extending the 15-mer peptides that originate from proteins classified as at least partly extracellular using the consensus sequence generated for each protein in step b) in the N- and C-terminal directions until the peptide length is between 25 to 35 amino acids, thereby creating the third set of peptides comprising at least one of MHC class II binding peptides or extended MHC class II binding peptides; or
ii. for each 15-mer peptide, determining the corresponding full-length variant protein sequence of step a) with the highest sequence identity to the consensus sequence generated in step b) and extending each 15-mer peptide with the determined corresponding full-length protein sequence that flanks the 15-mer peptide sequence to create one or more mosaic protein sequences, thereby creating the third set of peptides comprising at least one of MHC class II binding peptides or mosaic protein sequences.

2. The method according to claim 1, further comprising the steps of:
between steps b) and c), creating an 8-11-mer peptide set comprising at least one unique 8-, 9-, 10- or 11-mer peptide sequences for all protein sequences;
between steps c) and d), predicting MHC class I binding for each unique 8-11-mer peptide, for at least one MHC class I allele;
wherein step e) further comprises adding to the first set of selected peptides the unique 8-11-mer peptides that are predicted to bind to at least one HLA allele with a minimum binding score,
and wherein step f) comprises combining data describing
i. the first set of selected peptides;
ii. the corresponding MHC class I and class II alleles predicted to bind each peptide in the first set of selected peptides; and
iii. MHC class I and class II allele frequencies in a target population.

3. The method according to claim 1, further comprising a step i) of validating the immunogenicity of one or more peptides from the third set of peptides, such as by at least one of an in vivo assay, an in vitro assay or by database lookup, thereby generating a second set of validated peptides, and repeating steps f) to g) using said second set of validated peptides.

4. (canceled)

5. The method according to claim 1, wherein said target population is a human target population.

6. The method according to claim 1, wherein said MHC class II allele frequencies in said target population comprise at least one of HLA-DP, HLA-DQ or HLA-DR allele frequencies in a human target population.

7. The method according to claim 1, wherein said target population comprises at least two different species.

8. The method according to claim 1, further comprising a step of in silico prediction of the 3-dimensional folding properties of one or more of the MHC class II binding peptides aid/or extended MHC class II binding peptides in the third set of peptides.

9. The method according to claim 1, wherein the provided computer-readable list of protein sequences of step a) comprises at least one of unique 8-11-mer or 15-mer peptide sequences.

10. The method according to claim 1, wherein protein sequences from a protein that is at least partly extracellular and which is known to not be important outside the cell for establishment or maintenance of an infection are removed from said computer-readable list provided in step a).

11. The method according to claim 1, wherein the target pathogen is selected from the group consisting of a bacteria, a fungus, a virus, a protozoa and a worm.

12. The method according to claim 1, wherein the number of said variants of said target pathogen is 5 or more.

13. The method according to claim 1, wherein the multiple alignment of step b) is performed using a multiple sequence alignment method.

14. The method according to claim 2, wherein the 8-11-mer peptides or the 15-mer peptides are digitally stored with origin strain information for use in step g).

15. (canceled)

16. The method according to claim 2, wherein predicting MHC class I binding for each unique 8-11-mer peptide or predicting MHC class II binding for each unique 15-mer peptide is performed using an algorithm selected from the list consisting of NetMHCpan, MHCSeqNet, NetMHC, NetMHCcons, PickPocket and MHCflurry.

17. (canceled)

18. The method according to claim 2, wherein the predicted MHC class I binding of each peptide is digitally stored with origin strain information and digitally formatted for use in step g)); or wherein the predicted MHC class II binding of each peptide is digitally stored with origin strain information and digitally formatted for use in step g).

19. (canceled)

20. The method according to claim 1, wherein the minimum binding score of step e) is defined as a minimum output score threshold, a minimum affinity threshold, a minimum rank threshold or a combination thereof.

21. The method according to claim 1, wherein the second set of selected peptides of step g) is generated using the PopCover algorithm or is stored with all relevant meta-data in an independent digital table or database.

22. (canceled)

23. The method according to claim 1, wherein if the extension of the 15-mer peptide of step 10) i. in the C- or N-terminal direction reaches the end of the corresponding protein consensus sequence, the extension is continued at the opposite terminal until the peptide length is between 25 to 35 amino acids.

24. The method according to claim 1, wherein if two or more 15-mer peptide sequences of step h) ii. are from the same protein, overlap, and are different in an epitope defining sequence, only one of the peptides is embedded in the mosaic protein sequence.

25. The method according to claim 1, wherein the third set of peptides comprises or consists of peptide sequences each with a length between 8 to 35 amino acids.

26. A method for producing and formulating a vaccine, comprising:
1) performing the method according to claim 1; and
2) producing and formulating at least one peptide from the third set of at least one of peptides aid/or a nucleic acid sequence encoding said peptide.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A computer-readable medium comprising instructions which, when executed by a computer, cause the computer to carry out the method according to claim 1.

42. (canceled)

43. A data processing system comprising a processor configured to perform the method according to claim 1.

44. A composition comprising one or more peptides or one or more nucleic acids encoding said one or more peptides designed using to the method according to claim 1.

45. A pharmaceutical composition comprising one or more peptides or one or more nucleic acids encoding said one or more peptides designed using to the method according to claim 1, and at least one of pharmaceutically acceptable diluent, carrier or excipient.

46. (canceled)

47. (canceled)

48. A method for treating and/or preventing a disease in a subject in need thereof, the method comprising administering to the subject a pharmaceutical composition according to claim 45.

49. (canceled)


SEQUENCE LISTING (27 sequences)

SEQ ID NO: 1 - protein, 27 aa, synthetic construct
  REGION 1..27: note = Consensus sequence
  REGION 1..27: note = misc_feature - Consensus sequence
MSLLTEVETPTRNEWECRCSDSSDPLV

SEQ ID NO: 2 - protein, 566 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRASASSFFSRLNWLTHLNYSYPALNVTMPNNEQFDKLYIWGVHHPGTDKDQIFLYAQSSGRITVSTKRSQQAVIPNIGSRPRVRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 3 - protein, 469 aa, synthetic construct
MNPNQKIITIGSVSLTISTICFFMQIAILITTVTLHFKQYEFNSPPNNQVMLCEPTIIERNITEIVYLTNTTIEKEICPKLAEYRNWSKPQCPITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSIVSWSKNILRTQESECVCINGTCTVVMTDGPADGKADTKILFIEEGKIVHTSELSGSAQHVEECSCYPRYPGVRCVCRDNWKGSNRPIVDINIKDYSIVSSYVCSGLVGDTPRKNDSSSSSHCLGPNNEEGGHGVKGWAFDDGNDVWMGRTISETSRLGYETFKVPEGWSNTKSKLQINRQVIVDRGDRSGYSGIFSVEGKSCINRCFYVELIRGRKEETEVLWTSNSIVVFCGTSGTYGTGSWPDGADLNLMII

SEQ ID NO: 4 - protein, 15 aa, synthetic construct
VPDYASLRSLVASSG

SEQ ID NO: 5 - protein, 30 aa, synthetic construct
SNCYPYDVPDYASLRSLVASSGTLEFNNES

SEQ ID NO: 6 - protein, 15 aa, synthetic construct
GDKITFEATGNLVVP

SEQ ID NO: 7 - protein, 30 aa, synthetic construct
YWTIVKPGDKITFEATGNLVVPRGYFKIRS

SEQ ID NO: 8 - protein, 15 aa, synthetic construct
VKSQLKNNAKEIGNG

SEQ ID NO: 9 - protein, 30 aa, synthetic construct
MNKLFEKVKSQLKNNAKEIGNGCFKIYHKC

SEQ ID NO: 10 - protein, 15 aa, synthetic construct
NAELLVALENQHTID

SEQ ID NO: 11 - protein, 30 aa, synthetic construct
KIDLWSYNAELLVALENQHTIDLTDSEMNK

SEQ ID NO: 12 - protein, 15 aa, synthetic construct
TDTIKSWRNNILRTQ

SEQ ID NO: 13 - protein, 30 aa, synthetic construct
FIYNGRLTDTIKSWRNNILRTQESECVCIN

SEQ ID NO: 14 - protein, 15 aa, synthetic construct
FAPFSKDNSIRLSAG

SEQ ID NO: 15 - protein, 30 aa, synthetic construct
PQCPITGFAPFSKDNSIRLSAGGDIWVTRE

SEQ ID NO: 16 - protein, 15 aa, synthetic construct
AASYKIFKIEKGKVT

SEQ ID NO: 17 - protein, 30 aa, synthetic construct
TDGPADGAASYKIFKIEKGKVTHTSELSGS

SEQ ID NO: 18 - protein, 15 aa, synthetic construct
SYKIFRIEKGKIIKS

SEQ ID NO: 19 - protein, 30 aa, synthetic construct
GPADGKASYKIFRIEKGKIIKSSELSGSAQ

SEQ ID NO: 20 - protein, 9 aa, synthetic construct
YQKRMGVQM

SEQ ID NO: 21 - protein, 9 aa, synthetic construct
FMYSDFHFI

SEQ ID NO: 22 - protein, 9 aa, synthetic construct
GINDRNFWR

SEQ ID NO: 23 - protein, 9 aa, synthetic construct
TQIQTRRSF

SEQ ID NO: 24 - protein, 9 aa, synthetic construct
ITFMQALQL

SEQ ID NO: 25 - protein, 9 aa, synthetic construct
SLLTEVETY

SEQ ID NO: 26 - protein, 9 aa, synthetic construct
KTRPILSPL

SEQ ID NO: 27 - protein, 9 aa, synthetic construct
YRYGFVANF